(19) United States
(12) Patent Application Publication
(10) Pub. No.: US 2023/0349922 A1
(43) Pub. Date: Nov. 2, 2023

(54) H2 Blockers Targeting Liver Macrophages for the Prevention and Treatment of Liver Disease and Cancer

(71) Applicants: Université de Strasbourg, Strasbourg (FR); INSERM (INSTITUT NATIONAL DE LA SANTÉ ET DE LA RECHERCHE MÉDICALE), PARIS (FR)

(72) Inventors: Thomas Baumert, Freiburg (DE); Emilie Crouchet, Duppigheim (FR)

(73) Assignees: Université de Strasbourg, Strasbourg (FR); Institut National de la Santé et de la Recherche Médicale, Paris (FR)

(21) Appl. No.: 18/020,450

(22) PCT Filed: Aug. 11, 2021
(86) PCT No.: PCT/EP2021/072341

§ 371 Date: Feb. 9, 2023

(30) Foreign Application Priority Data
Aug. 11, 2020 (EP) 20190514.8

(51) Int. Cl. G01N 33/68; A61K 31/506

(52) U.S. Cl. CPC G01N 33/6893; A61K 31/506; G01N 2500/10

(57) ABSTRACT

The present invention provides Histamine Receptor 2 antagonists and pharmaceutical compositions thereof for use in the treatment or prevention of liver disease, including liver fibrosis and hepato-biliary cancers. The present invention also relates to methods for identifying candidate compounds that are useful in the treatment or prevention of liver disease, including liver fibrosis and hepato-biliary cancers.

### RELATED PATENT APPLICATION

The present application claims priority to European Patent Application number EP 20 190 514, which was filed on Aug. 11, 2020. The European patent application is incorporated herein by reference in its entirety.

### BACKGROUND OF THE INVENTION

Progressive liver fibrosis, caused by viral (hepatitis B and hepatitis B viruses—HBV, HCV) or metabolic (alcoholic and non-alcoholic steatohepatitis [NASH]) etiologies frequently leads to highly lethal cirrhosis and hepatocellular carcinoma (HCC), a leading cause of cancer death globally (Bray et al., Cancer J. Clin., 2018, 68: 394-424). Advanced liver fibrosis has been shown to be the key risk factor for HCC in NASH with no approved treatment options (Hagström et al., J. Hepatol., 2017, 67: 1265-1273). Moreover, in advanced fibrosis, HCC risk persists despite viral cure (Kanwal et al., Hepatol. 2019, Nov. 1. doi: 10.1002/hep.31014). HCC incidence and death rates have sharply increased compared to other cancer sites (Bray et al., Cancer J. Clin., 2018, 68: 394-424). Given the extremely high prevalence of advanced liver fibrosis and cirrhosis, which are estimated to affect approximately 1-2% of the world population (Tsochatizis et al., Lancet, 2014, 383: 1749-1761), there is a major unmet medical need for chemoprevention of liver disease progression towards cancer development. While chemoprevention has the potential to significantly impact the prognosis of patients with chronic diseases by reducing lethal complications, the development of effective chemopreventive drugs has been a daunting task as evidenced by the absence of approved therapies or of drugs providing a significant survival benefit (Fujiwara et al., J. Hepatol., 2018, 68: 526-549).

The present Inventors have previously identified a pan-etiology 186-gene prognostic liver signature (PLS) in diseased liver tissues, which robustly predicts liver disease progression and carcinogenesis in multiple patients cohorts (Goossens et al., J. Am. Gastroenterol., 2016, 14: 1619-1628; Hoshida et al., N. Engl. J. Med., 2008, 359: 1995-2004; Hoshida et al., Cancer Res., 2013, 69: 7385-7392; King et al., Gut, 2015, 64: 1296; Nakagawa et al., Cancer Cell, 2016, 30: 879-890; WO 2016/174130) as well as in animal models (Fuchs et al., Hepatol., 2014, 59: 1577-1590; Nakagawa et al., Cancer Cell, 2016, 30: 879-890; Ono et al., Hepatol., 2017, 66: 1344-1346). They then developed PLS cell-based models (WO 2016/174130), the clinical relevance of which was confirmed by highly similar transcriptome dysregulation in the cell culture model and the diseased liver of clinical cohorts with corresponding liver disease etiologies. The cPLS systems offer opportunities to interrogate the mechanisms of liver disease progression as well as evaluate cancer preventive strategies for each of the major liver cancer etiologies.

### SUMMARY OF THE INVENTION

Combining single-cell RNA-Seq analyses of patient liver tissues with perturbation studies, the present Inventors uncovered HRH2⁺ (histamine receptor H2) liver macrophages as a novel, hitherto unrecognized nizatidine target for treatment of liver disease (including hepatocarcinoma (HCC)). Furthermore, the functional data they obtained on macrophages using other H2 blockers (e.g., Famotidine, Ranitidine, Icotidine, Etintidine, Sufotidine, Roxatidine, Lafuditine, Oxmetidine, and Cimetidine) suggest a class-effect of histamine 2 receptor antagonists.

Consequently, in one aspect, the present invention provides a method of identifying an agent useful for the treatment or prevention of liver disease, the method comprising steps of:
providing a candidate compound; and
identifying the candidate compound as an agent useful for the treatment or prevention of liver disease if the candidate compound modulates the activity and/or function of a histamine 2 receptor.
   In particular, the candidate compound is identified as an agent useful for the treatment or prevention of a liver disease, if the candidate compound modulates the activity and/or function of a histamine 2 receptor on macrophages, such as liver macrophages.

In certain embodiments, the candidate compound is a histamine 2 receptor antagonist (H2 antagonist).

In certain embodiments, the candidate compound is a H2 antagonist in liver macrophages and/or in hepatocytes and/or in hepatocellular carcinoma cells.

In a related aspect, the present invention provides a method of identifying an agent useful for the treatment or prevention of liver disease, the method comprising steps of:
providing a candidate compound, wherein the candidate compound is a H2 antagonist; and
identifying the candidate compound as an agent useful for the treatment or prevention of liver disease if the candidate compound modulates the inflammatory profile of liver macrophages and/or of hepatocytes and/or hepatocellular carcinoma cell lines.

In certain embodiments, the candidate compound modulates the inflammatory profile of liver macrophages and/or of hepatocytes and/or hepatocellular carcinoma cell lines if the candidate compound decreases the overexpression of at least one pro-inflammatory cytokine or of at least one pro-fibrotic cytokine or soluble expression factor in liver macrophages and/or of hepatocytes and/or hepatocellular carcinoma cell lines. The at least one pro-inflammatory cytokine may be selected from the group consisting of IL6, IL1-α, IL1-β, IL-18, CCl2, CCL5, CXCL1, CXCL2, CXCL5, and TNF-α; and the at least one pro-fibrotic cytokine or soluble expression factor may be selected from the group consisting of TGF-β, PDGF, and MMP9.

In yet another related aspect, the present invention provides a method of identifying an agent useful for the treatment or prevention of liver disease, the method comprising steps of:
providing a candidate compound, wherein the candidate compound is a H2 antagonist; and
identifying the candidate compound as an agent useful for the treatment or prevention of liver disease if the candidate compound:
(a) decreases the expression of phosphorylated CREB1 and/or the expression of CREB5 in liver macrophages and/or hepatocytes and/or hepatocellular carcinoma cell lines; and/or
(b) decreases the expression of CLEC5A; and/or
(c) decreases the expression of SIGLEC-10 in liver macrophages; and/or
(d) decreases the expression of phosphorylated CREB1 and/or the expression of CREB5 in a human liver cancer cell line.

In certain embodiments, the human liver cancer cell line is a Huh-7 derived cell line, in particular which models certain pathways of a liver macrophage.

In certain embodiments, the candidate compound is a selective H2 antagonist.

In certain embodiments of the methods provided herein, the candidate compound is be selected from the group consisting of proteins, peptides, peptidomimetics, peptoids, polypeptides, saccharides, steroids, RNA agents (such as SiRNAs), antibodies, ribozymes, antisense oligonucleotides, and small molecules.

In certain embodiments of the methods provided herein, the liver disease is selected from the group consisting of acute liver failure, liver fibrosis, alcohol-related liver disease, fatty liver disease (NASH, NAFLD), autoimmune liver disease, cirrhosis, genetic liver diseases, hepatitis and hepato-biliary cancers (such as hepatocellular carcinoma or HCC, and cholangio cellular carcinoma or CC).

In another aspect, the present invention provides a H2 antagonist, a chemical derivative thereof, a prodrug thereof, a pharmaceutically acceptable salt thereof, or a solvate thereof, for use in a method of treatment or prevention of liver disease in a subject.

In certain embodiment, the H2 antagonist has been identified using a method described herein.

In certain embodiments, the prodrug is a liver-targeted prodrug.

In certain embodiments, the H2 antagonist, chemical derivative thereof, prodrug thereof, pharmaceutically acceptable salt thereof, or solvate thereof is formulated with a liver-targeted drug carrier.

In certain embodiments, the H2 antagonist is a small molecule selected from the group consisting of Bisfentidine, Burimamide, Cimetidine, Dalcotidine, Donetidine, Ebrotidine, Etintidine, Famotidine, Icotidine, Impromidine Lafutidine, Lamtidine, Lavoltidine (Loxtidine), Lupitidine, Metiamide, Mifentidine, Niperotidine, Nizatidine, Osutidine, Oxmetidine, Pibutidine, Ramixotidine, Ranitidine, Ranitidine bismuth citrate, Roxatidine, Sufotidine, Tiotidine, Tuvatidine, Zaltidine, Zolantidine, AH-18801, AH-21201, AH-21272 SKF-93828, SKF-93996, AY-29315, BL-6341A (BMY-26539), BL-6548 (ORF-17910), BMY-25271, BMY-25368 (SKF-94482), BMY-25405, D-16637, DA-4634, FCE-23067, FRG-8701, FRG-8813, HB-408, HE-30-256, ICI-162846, ICIA-5165, IT-066 L-643441, L-64728, NO-794, ORF-17578 (BL-6217), RGW-2568, SR-58042, TAS, YM-14471, Wy-45086, Wy-45253, and Wy-45662, Wy-45727.

In certain embodiments, the H2 antagonist is Oxmetidine.

In certain embodiments, the liver disease is selected from the group consisting of acute liver failure, liver fibrosis, alcohol-related liver disease, fatty liver disease (NASH, NAFLD), autoimmune liver disease, cirrhosis, genetic liver diseases, hepatitis and hepato-biliary cancers (such as hepatocellular carcinoma (HCC), or cholangiocarcinoma (CCA)).

In yet another aspect, the present invention provides a pharmaceutical composition for use in the treatment or prevention of liver disease, the pharmaceutical composition comprising a H2 antagonist, a chemical derivative thereof, a prodrug thereof, a pharmaceutically acceptable salt thereof, or a solvate thereof as defined herein, and at least one pharmaceutically acceptable excipient.

In certain embodiments, the pharmaceutical composition is for use in the treatment or prevention of a liver disease selected from the group consisting of acute liver failure, liver fibrosis, alcohol-related liver disease, fatty liver disease (NASH, NAFLD), autoimmune liver disease, cirrhosis, genetic liver diseases, hepatitis and liver cancer (HCC, CCA).

These and other objects, advantages and features of the present invention will become apparent to those of ordinary skill in the art having read the following detailed description of the preferred embodiments.

### BRIEF DESCRIPTION OF THE DRAWING

FIG. 1. Nizatidine Reverses the Cellular Prognostic Liver Signature (PLS) by Inhibiting the HRB2/CREB Signaling Pathways. (A) Nizatidine and other HRH2 blockers reverse the poor prognosis status of the prognostic liver signature (PLS) in the cell-based cPLS system. Heatmaps show PLS global status (top) and PLS poor- and good-prognosis gene expression (bottom). (B) Immunodetection of HRH2 in Huh7.5.1^{dif} cells by immunofluorescence (top) and flow cytometry analysis (bottom). HRH2 is shown in magenta (Alexa Fluor™ 647) and nuclei in blue (DAPI). CTRL=cells incubated with AF 647-labelled secondary antibody. Scale bar: 10 μm. Fluorescent imaging was performed using an Axio Observer Z1 microscope. (C) PLS assessment in the cell-based model upon perturbation of the HRH2/cAMP/PKA axis (histamine 10 μM, 8-CPT cAMP 100 μM and H89 10 μM). PLS induction was determined by GSEA analysis using “mock” non-treated cells as reference. Results are from two experiments performed in triplicate. (D) Intracellular levels of cAMP were assessed by ELISA. Results are from two experiments performed in triplicate (mean±s.e.m; * denotes p<0.05). (E) Expression of histidine decarboxylase (HDC) in Huh7.5.1^{dif} cells analyzed by qRT-PCR. Results are expressed in %±sem compared to Mock cells and normalized to GAPDH mRNA. Results are from 3 independent experiments in triplicate. * denotes p<0.05. (F) Nizatidine inhibits HCV-mediated CREB1 activation and decreases CREB5 expression. Western blot analysis of phospho-CREB1 (pCREB1) (Ser133), total CREB1 and total CREB5 in Huh7.5.1^{dif} cells. NT=non-treated. Results are representative of one out of three experiments. (G) CREB5 is a driver of the HCV-induced PLS. CREB5 KO was performed as described in the Examples sections. Single guide RNA (sgCTRL) targeting GFP was used as a control. Heatmaps show PLS global status (upper part) and PLS poor- and good-prognosis gene expression (bottom part) as described. FDR: false discovery rate.

FIG. 2. Genetic Loss-of-function Studies Conform a Functional Role of HRH2 in Hepatocarcinogenesis. (A-B) HRH2 KO decreases cancer cell proliferation in cell culture. (A) HRH2 KO validation at genetic level using T7 endonuclease assay. (B) Effect of HRH2 KO on cancer cell proliferation. EdU-incorporation assay by FACS showing %+/−SD of proliferative EdU-positive cell from 3 independent experiments in CTRL and HRH2 KO cells (n=8; ** p<0.01, two-tailed Mann-Whitney U test). (C) Effect of HRH2 KO on cancer cell apoptosis induced by oxidative stress (H₂O₂). Cleaved caspase 3 is shown in green. Nuclei were counterstained in blue (DAPI). Scale bar, 100 μM. Graph shows integrated cleaved caspase 3 intensity/total cell number from 2 independent experiments (n=12; ** p<0.01, two-tailed Mann-Whitney U test) measured using Celigo Cytometer. Western blot analysis of cleaved- and total caspase 3 is shown. (D-F) Effect of HRH2 knock-down on cancer cell proliferation. (D) siRNA efficacy was assessed by measuring mRNA by qRT-PCR. (E) EdU-incorporation assay by FACS showing %+/−SD of proliferative EdU-positive cell from 3 independent experiments in cell transfected with siCTRL and siHRH2 (n=6; ** p<0.01, two-tailed Mann-Whitney U test). (F) Cell proliferation was assessed daily in Huh7.5.1 transfected with a siCTRL or a siHRH2 by cell counting (TC20 Automated Cell Counter). Two representative and independent experiments are shown. (G) Full cPLS induction is impaired by HRH2 KO. PLS induction was determined by GSEA analysis using “Mock” non-infected cells as reference.

FIG. 3. ScRNA-Seq Analyses of Patient Liver Tissue Uncover Pro-Inflammatory Liver Macrophages as Nizatidine Target. (A) t-SNE map of single-cell transcriptomes from normal liver tissue of donors without history of chronic liver disease highlighting the main liver cell compartments. Cells sharing similar transcriptome profiles are grouped by clusters and each dot represents one cell. Expression t-SNE map of HRH2, CLEC5A, CD163L1 and MARCO are shown. The color bar indicates log 2 normalized expression. (B) t-SNE map of single-cell transcriptomes from normal liver tissue of donors without history of chronic liver disease highlighting the main liver cell compartments. Data extracted from MacParland et al. (Nature Commun., 2018, 9: 4383). Cells sharing similar transcriptome profiles are grouped by colors and each dot represents one cell. Arrows indicate macrophage compartment. Expression t-SNE map of HRH2 and MARCO are shown. (C-G) Perturbation of gene expression by nizatidine in liver tissue from patient with chronic liver disease and HCC identifies liver macrophages as therapeutic target. CD45⁺ leucocytes from patient liver tissue were enriched by flow cytometry and were treated with nizatidine or vehicle control (DMSO). Single cells were then sorted and analyzed as described in the Examples section below. (C) t-SNE map of single-cell transcriptomes showing control (blue) and nizatidine-treated cells (yellow), the t-SNE map indicating the main cell compartments (MAFB=macrophages; CD8=T lymphocytes). (D) Expression t-SNE map of HRH2, macrophage markers, and Siglec-10 (related to immune checkpoint) are shown. The color bar indicates log 2 normalized expression. (E-F) GSEA for differentially expressed genes between nizatidine-treated macrophages and CTRL macrophages depicted in panel A. (E) Normalized Enrichment Score (NES) of genes related to macrophage activation (classical M1 vs alternative M2). (E) NES of the pathways significantly enriched after nizatidine treatment (FDR≤0.05). (G) Expression heatmap of differentially expressed genes in individual nizatidine- and control-treated macrophages with each row representing a single cell. Markers of inflammation, fibrogenesis/cancer and antigen presentation are shown. All genes are normalized by row from their own min to max (Log2 fold; p value≤0.05).

FIG. 4. HRH2 Expression in Primary Human Hepatocytes (PHH) and Macrophages. HRH2 expression in PHH and liver macrophages. (A) Upper panel: Immunodetection of HRH2 in PHH by flow cytometry. PHHs freshly isolated from patient liver tissue were stained for HRH2 and CK18 (hepatocyte marker) or isotypes CTRL. Lower panel: Immunofluorescence staining of HRH2 in PHH in magenta (Alexa Fluor™ 647) and nuclei in blue (DAPI) (confocal microscopy). (B) HRH2 expression in patient derived-liver macrophages. Macrophages were purified from liver tissue of patient without history of chronic liver disease by serial centrifugations and stained with anti-CD68 antibody (macrophage marker; FITC, green) and anti HRH2 antibody (Alexa Fluor™ 647, magenta). Nuclei are counter stained in blue (DAPI) (epifluorescence microscopy). (C) Hepatocyte-macrophage cross-talk. PHH isolated from patient liver tissue were transfected with GalNac siRNA CTRL or targeting HRH2 expression and subjected to stress using conditioned medium (CM) from pro-inflammatory macrophages or control medium. HRH2 and CREB5 expression were analyzed by qRT-PCR (One representative experiment out of 2 performed in triplicate is shown).

FIG. 5. Effect of Nizatidine and HRH2 KO on Cytokine Expression in a Cell Culture Model for M1 Macrophages. (A) Representative picture of differentiated macrophages. To generate macrophage-like cells (MO), THP-1 cells were treated with PMA for 24 hours. To generate M1-polarized THP-1 macrophages, THP-1 cells were treated with PMA plus LPS and IFNγ. M1-polarized THP-1 were treated with nizatidine for 48 hours. (B) Gene expression of different cytokines was measured by qRT-PCR. Results are expressed as mean+s.e.m from 3 independent experiments performed in triplicate. * denotes p<0.05; ** denotes p<0.01; *** denotes p<0.001. (C) Representative picture of M1 differentiated patient-derived Kupffer cells. To generate M1-polarized macrophages, patient Kupffer cells were treated with LPS and IFNγ. (D) Gene expression of different cytokines was measured by qRT-PCR. Results are expressed as mean+s.d. from one experiment performed in duplicate. (E) IL6 expression in M1-polarized tumor associated macrophages (TAMs) isolated from different patient HCCs and treated with nizatidine. Results are expressed as mean+/−SD from one experiment performed in triplicate or quadruplicate. NT=non-treated (=100%). (F) HRH2 KO perturbates cytokine expression in pro-inflammatory macrophages. Left: HRH2 KO cells were generated using RNP technology. Validation of sgRNA targeting HRH2 in THP1 cells after clonal selection. KO efficacy was assessed at genetic level by T7 endonuclease assay. Control cells correspond to parental THP1 cell line. Clone 6 was selected for further analysis. Right: Pro-inflammatory cytokines and markers expression were analyzed by qRT-PCR (mean+/−SD n=3, one representative experiment out of two is shown).

FIG. 6. Effect of HRH2 Blocker Treatment on Inflammatory Cytokine Expression in THP1-derived Inflammatory Macrophages. (A) Experimental approach. To generate MO macrophages, THP-1 cells were treated with PMA for 24 hours. M1-polarized macrophages were generated by treatment of MO macrophages with LPS and IFN-7. M1-polarized macrophages were then treated with a panel of HRH2 blockers (10 μM) or DMSO as control for 72 hours. Expression of pro-inflammatory cytokines was assessed by qRT-PCR. (B-C) Expression of the pro-inflammatory cytokines (B) Interleukin 6 (IL6) and (C)C-C Motif Chemokine Ligand 2 (CCL2). Results are from at least two independent experiments performed in triplicate. Graphs show mean±s.e.m. Expression in M1 macrophages=100% (black dashed lines). *=p<0.05; **=p<0.01. For IL6, significance was determined using “M1+DMSO” as reference (blue dashed line).

FIG. 7. Dose-Response Studies on IL6 and CCL2 Expression in THP1-derived Inflammatory Macrophages. M1-polarized macrophages were treated with different concentrations of HRH2 blockers (the best candidates identified in FIG. 8) or DMSO as control (black) for 72 hours. (A-B) Expression of IL6 (A) and CCL2 (B) was assessed by qRT-PCR. Results are representative of one out of two independent experiments performed in triplicate. Graphs show mean±s.d. Expression in M1 macrophages=100%. Statistical significance was determined using the Holm-Sidak method, with alpha=0.05. Each row was analyzed individually, without assuming a consistent SD. (C) Analysis of cytotoxicity. Compounds toxicity was assessed on M1-polarized macrophages using a MTT assay. Graphs show mean±s.d. Mock=100%. Cell viability was calculated using a standard curve obtained by serial dilution of cells.

FIG. 8. The Huh7.5.1^{dif} Cell-Based System Recapitulates Transcriptional Dysregulations in Human Macrophages. (A-B) HRH2 knockdown leads to CREB5 downregulation in Huh7.5.1^{dif} cells similar to macrophages. Macrophages (A) and Huh7.5.1^{dif} cells (B) were transfected with siRNA CTRL or targeting HRH2 expression and activated using LPS+IFNγ or HCV-infection respectively. HRH2, CREB5 expression and the HCV viral load were analyzed by qRT-PCR. (Macrophage=three independent experiments n=8; Huh7.5.1^{dif} cells=two independent experiments n=6). (C) Nizatidine treatment decreases IL6 and TNF expression in Huh7.5.1^{dif} cells similar to macrophage. Huh7.5.1^{dif} cells were HCV infected and treated with nizatidine. IL6 and TNFα expression were analyzed by qRT-PCR. (two independent experiments n=8; * p<0.05; ** p<0.01; *** p<0.001).

FIG. 9. Proof-of-concept for Therapeutic Impact of Nizatidine in Patient-Derived HCC Spheroids. (A) Absent effect on cell viability in PHH, assessed 4 days after nizatidine treatment in 3D culture. Each experiment shows mean±SD in percentage compared to DMSO treated cells (n=4). (B) Nizatidine decreases HCC cell viability in a 3D patient-derived tumorspheroid model. HCC spheroids were generated from patient HCC tissues with different etiologies. Cell viability was assessed 4 days after treatment by measuring ATP levels. Each experiment shows mean±SD in percentage compared to DMSO treated spheroids (n=4). * p<0.05; ** p<0.01, unpaired t test. The pictures show representative image of patient-derived tumorspheroids (magnification X40). NASH=non-alcoholic steatohepatitis; ALD=alcoholic liver disease. Source data are provided as a Source Data file.

FIG. 10. Oxmeditine Decreases Inflammation and Improves Lipid Metabolism in a Cell Culture Model for M1 Macrophages. (A-B) Effect of Oxmetidine on gene set modulation (RNA-Seq) in a cell culture model for M1 macrophages. M1-polarized THP1 were treated with Oxmetidine or DMSO for 72 hours before performing RNA-Seq analysis. GSEA for differentially expressed genes between Oxmetidine-treated and CTRL macrophages was performed. The Normalized Enrichment Scores (NES) of the pathways (A=Reactome; B=Hallmark) significantly enriched after Oxmetidine treatment (FDR≤0.05) are shown. (C-D) Top 10 of the genes modulated by Oxmetidine treatment and comparison with Nizatidine treatment. (C) Gene expression modulation of the top 10 differentially expressed genes in Oxmetidine-treated M1 macrophages. All the genes are normalized by row from their own min to max (Log2 fold). (D). Gene expression modulation of the top 10 differentially expressed genes identified in C in nizatidine treated M1 macrophages. *** p<0.001; * p<0.05 (FDR). (E-F) Validation of gene expression of CD163 and CCL7 by qRT-PCR in Oxmetidine-treated M1 macrophages and comparison with Nizatidine treatment. M1-polarized THP1 were treated with Oxmetidine, Nizatidine or DMSO for 72 hours before performing qRT-PCR. Gene expression of different CD163 (E) and CCL7 (F) was measured by qRT-PCR and normalized by GAPDH mRNA. Results are expressed as % mean+s.d from one experiment performed in triplicate.

### DETAILED DESCRIPTION OF CERTAIN PREFERRED EMBODIMENTS

As mentioned above, the present invention relates to a method for the screening and identification of therapeutic agents useful in the treatment or prevention of liver disease; to such identified therapeutic agents or derivatives thereof; to their use in the treatment or prevention of liver disease, and to their liver-targeted delivery.

### I—Screening and Identification of Therapeutic Agents for the Prevention and Treatment of Liver Disease

The present inventors have demonstrated that nizatidine, a histamine 2 receptor (HRH2) blocker, inhibits liver inflammation, fibrosis and carcinogenesis, and have uncovered HRH2⁺ liver macrophages as a nizatidine target. Furthermore, the functional data they obtained in macrophages suggest a class-effect of histamine 2 receptor antagonists.

### Screening and Identification of Liver Disease Therapeutic and Chemopreventive Agents

Consequently, the present invention provides methods for identifying agents that are useful in the treatment or prevention of liver disease.

In certain embodiments, a method for identifying an agent useful for the treatment or prevention of liver disease according to the present invention comprises steps of: providing a candidate compound; and identifying the candidate compound as an agent useful for the treatment or prevention of liver disease if the candidate compound is a histamine 2 receptor antagonist.

The terms “histamine 2 receptor antagonist”, “H2 blocker”, “H2 antagonist”, “HRH2 blocker”, “HRH2 antagonist” and “H2RA” are used herein interchangeably. They refer to a molecule or agent that acts, for example, in cells in culture or in vivo, to reduce, decrease, diminish, or lessen a biological or physiological activity of the histamine 2-receptor (H2 receptor) elicited by histamine. These terms are meant to include any type of interaction that changes, modulates, influences the activity of the HRH2 molecule. In the context of the present invention, the H2 blocker may be any kind of molecule that interacts with HRH2 such that the interaction results in such an effect on HRH2 activity. Thus, the H2 blocker may be, for example, a reversible antagonist, an irreversible antagonist, an inverse agonist, a partial agonist, a conformational modulator, etc. As used herein, the term “reversible antagonist” refers to an antagonist capable of readily dissociating from the biologically active molecule with which it associates (here the H2 receptor), thereby forming a short-lasting or transient combination with the biologically active molecule. A reversible H2 antagonist is a competitive inhibitor of the action of histamine at the H2 receptors. As used herein, the term “irreversible antagonist” refers to an agonist, which forms a stable chemical bond with the biologically active molecule with which it associates (here the H2 receptor), thereby forming a long-lasting combination with the biologically active molecule. An irreversible H2 antagonist is a non-competitive inhibitor of the action of histamine at the H2 receptors.

In the context of the present invention, preferred H2 blockers are selective H2 antagonists which block H2 receptors, but do not have any or any significant activity in blocking histamine 1 receptors (H1 receptors). Thus, in certain preferred embodiments, a method for identifying an agent useful for the treatment or prevention of liver disease according to the present invention comprises steps of: providing a candidate compound and identifying the candidate compound as an agent useful for the treatment or prevention of liver disease if the candidate compound is a selective histamine 2 receptor antagonist.

A candidate compound may be identified as an agent useful for the treatment or prevention of liver disease if the candidate compound has been found to be a H2 antagonist through performance in classical preclinical screening tests for H2 antagonistic function (e.g., competition or receptor displacement assays). In particular, a candidate compound may be identified as an agent useful for the treatment or prevention of liver disease if the candidate compound has been demonstrated to function as reversible or irreversible antagonist in those screening models specifically dependent upon H2 receptor function but lacks significant histamine antagonist activity in those screening models dependent upon H1 receptor function. For example, this includes compounds that would be classified, as described by J. W. Black et al. (Nature, 1972, 236: 385-390) as H2 antagonists if assessed through testing with guinea pig spontaneously beating right atria in vitro assay and the rat gastric acid secretion in vivo assay, but shown to lack in significant H1 antagonist activity through testing with either the guinea pig ileum contraction in vitro assay or the rat stomach muscle contraction in vivo assay. In certain embodiments, candidate compounds identified as useful in the treatment or prevention of liver disease is a H2 antagonist that demonstrates no significant H1 activity at reasonable dosage levels in the above-mentioned H1 assays. Typically, reasonable dosage level is the lowest dosage level at which 90% inhibition of histamine, or 99% inhibition, is achieved in the above-mentioned H2 assays.

In other embodiments, a method for identifying an agent useful for the treatment or prevention of liver disease according to the present invention comprises steps of: providing a candidate compound, wherein the candidate compound is a histamine 2 receptor antagonist; and identifying the candidate compound as an agent useful for the treatment or prevention of liver disease if the candidate compound modulates the inflammatory profile of liver macrophages.

As used herein the term “liver macrophages” refers to cells that represent a key cellular component of the liver and are essential for maintaining tissue homeostasis and ensuring rapid responses to hepatic injury. Liver macrophages consist of self-sustaining, liver-resident phagocytes, known as Kupffer cells, and bone marrow-derived recruited monocytes, which quickly accumulate in the injured liver, and of other subsets and variants of liver macrophages, as recently reviewed by Bldriot and Ginhoux, Front. Immunol., 2010, 10: 2694, doi.org/10.3389/fimmu.2019.02694).

As used herein, the expression “to modulate the inflammatory profile of liver macrophages” refers to the ability of a candidate compound to decrease the overexpression of pro-inflammatory and/or pro-fibrotic cytokines and/or soluble expression factors in liver macrophages and/or changing the transcriptional profile. Examples of pro-inflammatory cytokines known to be involved in liver disease include, but are not limited to, interleukin 6 (IL6), interleukin 1 (IL1, including IL1-α and IL1-β), interleukin 18 (IL-18); chemokine ligand 2 (CCl2), chemokine ligand 5 (CCL5), chemokine (C-X-C motif) ligand 1 (CXCL1), chemokine (C-X-C motif) ligand 2 (CXCL2), chemokine (C-X-C motif) ligand 5 (CXCL5), tumor necrosis factor alpha (TNF-α), and interferon gamma (IFN-γ). Examples of pro-fibrotic cytokines or soluble expression factors known to be involved in liver disease include, but are not limited to, growth factor beta (TGF-0), platelet-derived growth factor (PDGF), MMP9 (matrix metalloproteinase 9), interleukin 10 (IL10) and interleukin 13 (IL13).

In the screening method disclosed herein, the candidate compound is identified as an agent useful for the treatment or prevention of liver disease if the candidate compound decreases the expression of pro-inflammatory cytokines, factors and markers listed above, or described as such in the literature. In certain preferred embodiments, the candidate compound decreases the expression of: IL6, CCl2 and/or CLEC5A.

In yet other embodiments, a method for identifying an agent useful for the treatment or prevention of liver disease according to the present invention comprises steps of: providing a candidate compound, wherein the candidate compound is a histamine 2 receptor antagonist; and identifying the candidate compound as an agent useful for the treatment or prevention of liver disease if the candidate compound decreases the expression of phosphorylated CREB1 (cAMP responsive element binding protein 1) and/or the expression of CREB5 (cAMP responsive element binding protein 5), which are both overexpressed in liver macrophages during liver injury. CREB1 and CREB5 are two key members of the CREB family, that have been found to be overexpressed in many solid tumors including HCC (Abramovitch et al., Cancer Res., 2004, 64: 1338-1346; He et al., Oncol., Lett., 2017, 14: 8156-8161; Steven et al., Oncotarget, 2016, 7: 35454-35465).

In still other embodiments, a method for identifying an agent useful for the treatment or prevention of liver disease according to the present invention comprises steps of: providing a candidate compound, wherein the candidate compound is a histamine 2 receptor antagonist; and identifying the candidate compound as an agent useful for the treatment or prevention of liver disease if the candidate compound decreases the expression of C-type lectin domain family 5 member A (CLEC5A). CLEC5A, which is overexpressed in pro-inflammatory macrophages, is known to be involved in signaling transduction and production of pro-inflammatory cytokines (Gonzilez-Dominguez et al., J. Leukoc. Biol., 2015, 98: 453-466). MARCO, the decreased expression of which has been reported to be associated with tumor progression and poor-diagnosis in human hepatocellular carcinoma (Sun et al., J. Gastroenterol. Hepatol., 2017, 32(5): 1107-1114), is under-expressed in immunoregulatory liver macrophages.

In still other embodiments, a method for identifying an agent useful for the treatment or prevention of liver disease according to the present invention comprises steps of: providing a candidate compound, wherein the candidate compound is a histamine 2 receptor antagonist; and identifying the candidate compound as an agent useful for the treatment or prevention of liver disease if the candidate compound decreases the expression of the receptor sialic-acid-binding Ig-like lectin 10 (SIGLEC-10) in liver macrophages. SIGLEC-10 is a recently uncovered immune checkpoint shown to inhibit effector functions of immune cells in cancer (Bârenwaldt and Laubli, Expert Opin. Ther. Targets, 2019, 23: 839-853).

### Candidate Compounds

Screening according to the present invention is generally performed with the goal of developing therapeutics useful in the treatment and/or prevention of liver disease, for example with the goal of developing liver fibrosis/cirrhosis/HCC chemopreventive drugs.

As will be appreciated by those of ordinary skill in the art, any kind of compounds can be tested using the inventive methods. A candidate compound may be a synthetic or natural compound; it may be a single molecule, or a mixture or complex of different molecules. In certain embodiments, a method of screening is used for testing one candidate compound or a few candidate compounds. In other embodiments, a screening method is used for screening collections or libraries of candidate compounds. As used herein, the term “collection” refers to any set of compounds, molecules or agents, while the term “library” refers to any set of compounds, molecules or agents that are structural analogs.

Collections of natural compounds in the form of bacterial, fungal, plant and animal extracts are available from, for example, Pan Laboratories (Bothell, WA) or MycoSearch (Durham, NC). Libraries of candidate compounds that can be screened using the methods of the present invention may be either prepared or purchased from a number of companies. Synthetic compound libraries are commercially available from, for example, Comgenex (Princeton, NJ), Brandon Associates (Merrimack, NH), Microsource (New Milford, CT), and Aldrich (Milwaukee, WI). Libraries of candidate compounds have also been developed by and are commercially available from large chemical companies, including, for example, Merck, Glaxo Welcome, Bristol-Meyers-Squibb, Novartis, Monsanto/Searle, and Pharmacia UpJohn. Additionally, natural collections, synthetically produced libraries and compounds are readily modified through conventional chemical, physical, and biochemical means. Chemical libraries can be prepared by traditional or automated synthesis, or proprietary synthetic methods (see, for example, DeWitt et al., Proc. Natl. Acad. Sci. U.S.A. 1993, 90:6909-6913; Zuckermann et al., J. Med. Chem. 1994, 37: 2678-2685; Carell et al., Angew. Chem. Int. Ed. Engl. 1994, 33: 2059-2060; Myers, Curr. Opin. Biotechnol. 1997, 8: 701-707). Candidate compounds may also be obtained by any other of the numerous approaches in combinatorial library methods known in the art, including peptoid libraries, spatially addressable parallel solid phase or solution phase libraries; synthetic library methods requiring deconvolution; the “one-bead one-compound” library method; and synthetic library methods using affinity chromatography selection.

Candidate compounds may be found within a large variety of classes of chemicals, including proteins, peptides, peptidomimetics, peptoids, polypeptides, saccharides, steroids, RNA agents (e.g., mRNA or siRNA), antibodies, ribozymes, antisense oligonucleotides, small molecules and the like. In certain embodiments, the screening methods of the invention are used for identifying compounds or agents that are small molecules. The term “small molecule”, as used herein, refers to any natural or synthetic organic or inorganic compound or factor with a low molecular weight. Preferred small molecules have molecular weights of more than 50 Daltons and less than 2,500 Daltons. More preferably, small molecules have molecular weights of less than 600-700 Daltons. Even more preferably, small molecules have molecular weights of less than 350 Daltons.

In certain embodiments, the candidate compounds to be tested using a screening method of the invention have been previously selected by transcriptome-based in silico drug screening using the PLS risk signature exhibited by cells of the cellular model system described in WO 2016/174130.

In certain preferred embodiments, the candidate compound to be tested is a known H2 blocker, in particular a known selective H2 antagonist (see below).

### Screening Methods

In a screening method according to the present invention, determining if a candidate compound decreases the overexpression of pro-inflammatory and/or pro-fibrotic cytokines and/or soluble expression factors in liver macrophages; or decreases the expression of phosphorylated CREB1 and/or the expression of CREB5 in liver macrophages; or decreases the expression CLEC5A; or decreases the expression of SIGLEC-10 in liver macrophages, may be performed using any suitable method known in the art that allows to assess the effect of a compound or agent on the expression of the marker of interest. Several examples of such methods are described in the Examples section below.

Alternatively, or additionally, the expression of phosphorylated CREB1 and/or the expression of CREB5 is measured in liver-derived cell lines, such as Huh7-derived cell lines or others. Indeed, the Inventors have shown that Huh7-derived cell lines model disease-causing phenotype of macrophages.

A method according to the present invention may, for example, include steps of: (a) contacting the candidate compound with liver macrophages that overexpress at least one of the markers of interest (pro-inflammatory and/or pro-fibrotic cytokines and/or soluble expression factors, phosphorylated CREB1, CREB5, CLEC5A, SIGLEC-10); (b) measuring the expression level of the at least one marker of interest; (c) comparing the expression level measured in (b) with the expression level of the at least one marker of interest measured in the same conditions but in the absence of the candidate compound; and (d) identifying the candidate compound as an agent useful for the treatment or prevention of liver disease if the expression level measured in (b) is lower than the expression level of the at least one marker of interest measured in the absence of the candidate compound.

One skilled in the art knows how to obtain liver macrophages that overexpress at least one of the markers of interest (Aizarani et al., Nature, 2019, 572: 199-204).

As used herein, the term “contacting the candidate compound with liver macrophages” typically includes, but is not limited to, mixing or incubating the liver macrophages with the candidate compound. Preferably, the step of contacting the candidate compound with liver macrophages is performed for a time and under conditions allowing the candidate compound to exert its effect. Generally, concentrations from about 1 μM to about 10 mM are used for screening. Preferred screening concentrations are between about 1 nM and about 100 μM.

In these methods, the levels of expression of the at least one marker may be assessed at the protein level or at the mRNA level. Methods for determining the level of expression of a marker at either the nucleic acid or protein level are well known in the art and include, but are not limited to, immunoblots (Western blots), Northern blots, enzyme linked immunosorbent assay (ELISA), immunoprecipitation, immunofluorescence, flow cytometry, immunohistochemistry, nucleic acid hybridization techniques, nucleic acid reverse transcription methods, and nucleic acid amplification methods.

Once the expression level of the at least one marker of interest has been determined, it is compared to the expression level of the same marker measured under the same conditions with the exception of the presence of the candidate compound. As known in the art, comparison of expression levels is preferably performed after the expression levels measured have been corrected for both differences in the amount of liver macrophages assayed and variability in the quality of the sample.

### Identification of Liver Disease Therapeutic and Chemopreventive Agents

Comparison of the expression level of the maker of interest measured in the presence of the candidate compound with the expression level of the maker of interest measured in the absence of the candidate compound allows to determine whether the candidate compound is an agent useful for the treatment or prevention of liver disease.

Thus, in a screening method of the invention, a candidate compound is identified as an agent useful for the treatment or prevention of liver disease if the candidate compound is a (selective) H2 antagonist, and optionally if the candidate compound has the ability to decrease the expression, in liver macrophages, of at least one of the following markers: pro-inflammatory and/or pro-fibrotic cytokines and/or soluble expression factors (such as IL6, IL1-α, IL1-β, IL-18, CCl2, CCL5, CXCL1, CXCL2, CXCL5, TNF-α, TGF-β, PDGF, MMP9, and the like); phosphorylated CREB1; CREB5; CLEC5A; and SIGLEC-10.

As used herein, the terms “decrease” and “lower” refer to a decrease (or reduction) of at least 5%, at least about 10%, at least about 20%, at least 25%, at least 30%, at least 40%, at least about 50%, at least about 75%, at least about 80%, at least about 100%, at least about 200% (i.e., 2-fold), or at least about 500% (i.e., 5-fold), or at least about 10,000% (i.e., 100-fold) or more of the level of expression in control conditions (i.e., in the absence of the candidate compound).

As used herein, the terms “increase” and “higher” refer to an increase (or augmentation) of at least 5%, at least about 10%, at least about 20%, at least 25%, at least 30%, at least 40%, at least about 50%, at least about 75%, at least about 80%, at least about 100%, at least about 200% (i.e., 2-fold), or at least about 500% (i.e., 5-fold), or at least about 10,000% (i.e., 100-fold) or more of the level of expression in control conditions (i.e., in the absence of the candidate compound).

In certain embodiments, a screening method of the invention further involves the use of one or more negative or positive control compounds. A positive control compound may be any molecule, agent, or drug that is known to decrease (or increase) the expression level of a marker of interest. A negative control compound is any molecule, agent, or drug that is known to have no effect on the expression level of a marker of interest. In such embodiments, the screening method further comprises a step of comparing the effects of the candidate compound on the expression level of the marker of interest to the effects (or absence thereof) of the positive or negative control compound. Such negative and positive control compounds are known in the art or may be identified by the methods described herein.

### Characterization of Candidate Liver Disease Therapeutic and Chemopreventive Agents

As will be recognized by one skilled in the art, reproducibility of a screening method according to the present invention may be tested by incubating liver macrophages with the same concentration of the same candidate compound. Additionally, since candidate compounds may be effective at different concentrations depending on the nature of the candidate compound, varying concentrations of the candidate compound may be tested. Generally, concentrations from about 1 μM to about 10 mM are used for screening. Preferred screening concentrations are between about 10 nM and about 100 μM. Furthermore, testing different concentrations of a candidate compound according to the methods of the invention allows the IC₅₀ value to be determined for that compound.

The present invention pertains to the combination of a screening method described herein with one or more additional screening assays. For example, when a screening method of the invention allows to identify a candidate compound as potentially useful as liver disease therapeutic or preventive agent, the efficacy of the candidate compound can be further confirmed ex vivo, e.g., in animal or human biopsy material or in vivo, e.g., in a whole animal model for liver disease.

Accordingly, it is within the scope of this invention to further use a candidate compound identified by a screening method described herein in an appropriate in vivo animal model and/or in ex vivo animal or human biopsy materials. For example, a compound identified as described herein can be used in an animal model to determine the efficacy, toxicity, or side effects of treatment with such a compound. Alternatively, a compound identified as described herein can be used in an animal model to determine the mechanism of action of such a compound.

As known in the art, once a candidate compound has been identified as an agent useful for the treatment or prevention of liver disease using a screening method of the present invention, the structure of the candidate compound may be modified with the goal of developing derivatives of the candidate compound that exhibit increased biological efficacy or display other desired properties. To this end, structure-activity relationship (SAR) studies may be conducted. The expression “increased biological efficacy”, as used herein, refers to any biological property (solubility, stability, affinity, efficiency, and the like) of the chemical derivative that is improved compared to the parent candidate compound.

The invention also pertains to the use of candidate compounds identified by a screening method described herein, or of chemical derivatives thereof, for pre-clinical and clinical assays.

The present invention further encompasses candidate compounds identified by a screening method described herein, or chemical derivatives thereof, for use in the treatment or prevention of liver disease (see below).

### II—Specific H2 Antagonists

In certain preferred embodiments, the candidate compounds to be tested in a screening method described above or to be used in a method of treatment or prevention of liver disease according to the present invention are selected among molecules and agents that are known as selective H2 antagonists. Today, selective H2 antagonists are mainly used to inhibit or block the secretion of gastric acid by binding to H2 receptors on parietal cells in the stomach, thereby inhibiting the binding and action of the endogenous ligand histamine. H2 antagonists are approved for short-term use in the treatment of uncomplicated gastroesophageal reflux disease (GERD), gastric or duodenal ulcers, gastric hypersecretion, and for mild to infrequent heartburn or indigestion. They also find application for ulcer prophylaxis and for the treatment of esophagitis, gastritis, and gastrointestinal hemorrhage.

Examples of suitable selective H2 antagonists for use in the context of the present invention include compounds which are described in U.S. Pat. Nos. 5,294,433 and 5,364,616 and references cited therein (each of these U.S. patents and references is incorporated herein by reference in its entirety).

For example, suitable selective H2 antagonists are disclosed in U.S. Pat. Nos.: 3,751;470; 3,876,647; 3,881,016; 3,891,764; 3,894,151; 3,897,444; 3,905,964; 3,910,896; 3,920,822; 3,932,443; 3,932,644; 3,950,333; 3,968,227; 3,971,786; 3,975,530; 3,979,398; 4,000,296; 4,005,205; 4,024,271; 4,034,101; 4,035,374; 4,036,971; 4,038,408; 4,056,620; 4,056,621; 4,060,621; 4,062,863; 4,062,967; 4,070,472; 4,072,748; 4,083,983; 4,083,988; 4,084,001; 4,090,026; 4,093,729; 4,098,898; 4,104,381; 4,104,472; 4,105,770; 4,107,319; 4,109,003; 4,112,104; 4,112,234; Re. 29,761; 4,118,496; 4,118,502; 4,120,966; 4,120,968; 4,120,972; 4,120,973; 4,128,658; 4,129,657; 4,133,886; 4,137,319; 4,139,624; 4,140,783; 4,145,546; 4,151,289; 4,152,443; 4,152,453; 4,153,793; 4,154,834; 4,154,838; 4,156,727; 4,157,347; 4,158,013; 4,160,030; 4,165,377; 4,165,378; 4,166,856; 4,166,857; 4,169,855; 4,170,652; 4,173,644; 4,181,730; 4,185,103; 4,189,488; 4,190,664; 4,191,769; 4,192,879; 4,197,305; 4,200,578; 4,200,760; 4,203,909; 4,210,652; 4,210,658; 4,212,875; 4,215,125; 4,215,126; 4,216,318; 4,218,452; 4,218.466; 4,219,553; 4,220,767; 4,221,737; 4,227,000; 4,233,302; 4,234,588; 4,234,735; 4,238,493; 4,238,494; 4,239,769; Re. 30,457; 4,242,350; 4,242,351; 4,247,558; 4,250,316; 4,252,819; 4,255,425; 4,255,440; 4,260,744; 4,262,126; 4,264,608; 4,264,614; 4,265,896; 4,269,844; 4,271,169; 4,276,297; 4,276,301; 4,279,819; 4,279,911; 4,282,213; 4,282,221; 4,282,224; 4,282,234; 4,282,363; 4,283,408; 4,285,952; 4,288,443; 4,289,876; 4,293,557; 4,301,165; 4,302,464; 4,304,780; 4,307,104; 4,308,275; 4,309,433; 4,309,435; 4,310,532; 4,315,009; 4,317,819; 4,318,858; 4,318,913; 4,323,566; 4,324,789; 4,331,668; 4,332,949; 4,333,946; 4,336,394; 4,338,328; 4,338,447; 4,338,448; 4,341,787; 4,342,765; 4,347,250; 4,347,370; 4,359,466; 4,362,728; 4,366,164; 4,372,963; 4,374,248; 4,374,251; 4,374,839; 4,374,843; 4,375,435; 4,375,472; 4,375,547; 4,379,158; 4,380,638; 4,380,639; 4,382,090; 4,382,929; 4,383,115; 4,385,058; 4,386,099; 4,386,211; 4,388,317; 4,388,319; 4,390,701; 4,394,508; 4,395,419; 4,395,553; 4,399,142; 4,405,621; 4,405,624; 4,407,808; 4,410,523; 4,413,130; 4,426,526; 4,427,685; 4,432,983; 4,433,154; 4,435,396; 4,438,127; 4,439,437; 4,439,444; 4,439,609; 4,440,775; 4,442,110; 4,443,613; 4,447,441; 4,447,611; Re. 31,588; 4,450,161; 4,450,168; 4,451,463; 4,452,985; 4,452,987; 4,458,077; 4,461,900; 4,461,901; 4,464,374; 4,465,841; 4,466,970; 4,467,087; 4,468,399; 4,470,985; 4,471,122; 4,474,790; 4,474,794; 4,476,126; 4,481,199; 4,482,552; 4,482,563; 4,482,566; 4,485,104; 4,490,527; 4,491,586; 4,492,711; 4,493,840; 4,496,564; 4,496,571; 4,499,101; 4,500,462; 4,501,747; 4,503,051; 4,507,296; 4,507,485; 4,510,309; 4,510,313; 4,514,408; 4,514,413; 4,515,806; 4,518,598; 4,520,025; 4,521,418; 4,521,625; 4,522,943; 4,523,015; 4,524,071; 4,525,477; 4,526,973; 4,526,995; 4,528,375; 4,528,377; 4,528,378; 4,529,723; 4,529,731; 4,536,508; 4,537,779; 4,537,968; 4,539,207; 4,539,316; 4,540,699; 4,543,352; 4,546,188; 4,547,512; 4,548,944; 4,550,118; 4,551,466; 4,558,044; 4,558,128; 4,559,344; 4,560,690; 4,564,623; 4,567,176; 4,567,191; 4,570,000; 4,571,394; 4,571,398; 4,574,126; 4,578,388; 4,578,459; 4,578,471; 4,584,384; 4,585,781; 4,587,254; 4,588,719; 4,588,826; 4,590,192; 4,590,299; 4,595,683; 4,595,758; 4,596,811; 4,599,346; 4,600,720; 4,600,779; 4,600,780; 4,604,465; 4,607,105; 4,607,107; 4,608,380; 4,612,309; 4,613,596; 4,613,602; 4,621,142; 4,622,316; 4,632,927; 4,632,993; 4,634,701; 4,638,001; 4,639,442; 4,639,523; 4,643,993; 4,644,006; 4,645,841; 4,647,559; 4,649,141; 4,649,145; 4,649,150; 4,650,893; 4,652,572; 4,652,575; 4,656,176; 4,656,180; 4,657,908; 4,659,721; 4,663,331; 4,665,073; 4,666,932; 4,668,673; 4,668,786; 4,670,448; 4,673,747; 4,675,406; 4,681,883; 4,683,228; 4,687,856; 4,692,445; 4,692,456; 4,692,531; 4,694,008; 4,696,933; 4,699,906; 4,699,915; 4,704,388; 4,705,873; 4,710,498; 4,716,228; 4,722,925; 4,727,081; 4,727,169; 4,728,655; 4,732,980; 4,738,960; 4,738,969; 4,738,983; 4,742,055; 4,743,600; 4,743,692; 4,745,110; 4,746,672; 4,748,164; 4,748,165; 4,749,790; 4,758,576; 4,760,075; 4,762,932; 4,764,612; 4,767,769; 4,769,473; 4,772,704; 4,777,168; 4,777,179; 4,788,184; 4,788,187; 4,788,195; 4,795,755; 4,806,548; 4,808,569; 4,814,341; 4,816,583; 4,837,316; 4,847,264; 4,851,410; 4,871,765; 4,886,910; 4,886,912; 4,894,372; 4,904,792; 4,912,101; 4,912,132; 4,937,253; 4,952,589; 4,952,591; 4,957,932; 4,965,365; 4,972,267; 4,988,828; 5,008,256; 5,021,429; 5,025,014; 5,037,837; 5,037,840; 5,047,411.

Selective H2 antagonists also include compounds described in the European Patent Applications Nos.: 7,326; 10,893; 17,679; 17,680; 29,303; 31,388; 32,143; 32,916; 49,049; 50,407; 57,227; 67,436; 73,971; 74,229; 79,297; 80,739; 86,647; 89,765; 103,503; 103,390; 104,611; 105,703; 112,637; 122,978; 134,096; 141,119; 141,560; 156,286; 169,969; 171,342; 172,968; 173,377; 178,503; 180,500; 181,471; 186,275; 204,148; 213,571; 277,900; 355,612; 417,751; 445,949; 454,449; 454,469.

Other selective H2 antagonists also include compounds described in U.K. Patent Applications Nos: 1,341,590; 1,531,237; 1,565,647; 1,574,214; 2,001,624; 2,067,987; 2,094,300; 2,117,769; 2,124,622; 2,146,331; 2,149,406; 2,162,174; 2,209,163; in Belgian Patent Applications Nos.: 857,218; 857,219; 866,155; 884,820; 892,350; 905,235; 1,000,307; in German Patent Applications Nos.: 3,044,566; 3,341,750; 3,644,246; in French Patent Applications Nos.: 2,515,181; 2,531,703; in Spanish Patent Applications Nos.: 85-06,610; 86-05,244; in Dutch Patent Application No. 88-02,089; in South African Patent Application No. 83-05,356; in Japanese Patent Applications Nos.: 53/005,180; 54/106,468; 55/053,247; 55/115,860; 55/115,877; 56/135,479; 57/054,177; 57/165,348; 57/169,452; 58/015,944; 58/072,572; 58/072,573; 58/090,569; 59/007,172; 59/010,582; 59/093,050; 59/093,051; 59/190,973; 60/197,663; 60/226,180; 60/228,465; 60/237,082; 61/063,665; 61/063,676; 61/115,072; 62/005,969; 62/126,169; 63/122,679; 63/183,563; 02/000,178; 02/056,449; 03/251,571.

Specific examples of suitable selective H2 antagonists include, but are not limited to, Bisfentidine, Burimamide, Cimetidine, Dalcotidine, Donetidine, Ebrotidine, Etintidine, Famotidine, Icotidine, Impromidine Lafutidine, Lamtidine, Lavoltidine (Loxtidine), Lupitidine, Metiamide, Mifentidine, Niperotidine, Nizatidine, Osutidine, Oxmetidine, Pibutidine, Ramixotidine, Ranitidine, Ranitidine bismuth citrate, Roxatidine, Sufotidine, Tiotidine, Tuvatidine, Zaltidine, Zolantidine, AH-18801, AH-21201, AH-21272 SKF-93828, SKF-93996, AY-29315, BL-6341A (BMY-26539), BL-6548 (ORF-17910), BMY-25271, BMY-25368 (SKF-94482), BMY-25405, D-16637, DA-4634, FCE-23067, FRG-8701, FRG-8813, HB-408, HE-30-256, ICI-162846, ICIA-5165, IT-066 L-643441, L-64728, NO-794, ORF-17578 (BL-6217), RGW-2568, SR-58042, TAS, YM-14471, Wy-45086, Wy-45253, Wy-45662, and Wy-45727.

In certain embodiments, the selective H2 antagonist is Cimetidine (N″-cyano-N-methyl-N″-[2-[[5-methyl-1H-imidazol-4-yl)methyl]thio]ethyl]guanidine), which is described in U.K. Patent No. 1,397,426, U.S. Pat. Nos. 3,950,333, 4,024,271. Cimetidine is marketed as the hydrochloride salt (TAGAMET™) and is used in the treatment of duodenal gastric, recurrent and stomal ulceration, and reflux esophagitis and in the management of patients who are at high risk of hemorrhage of the upper gastro-intestinal tract. The selective H2 antagonist may alternatively be Burimamide (1-[4-(1H-imidazol-5-yl)butyl]-3-methylthiourea) or Metiamide (N-Methyl-N″-(2-{[(5-methyl-1H-imidazol-4-yl)methyl]sulfanyl}ethyl) thiourea), which were first developed by scientists at Smith, Kline & French (now GlaxoSmithKline) in their intent to develop a histamine antagonist for the treatment of peptic ulcers, and ultimately led to the development of Cimetidine. Other H2 antagonists may be found among the substituted thioalkyl-, aminoalkyl- and oxyalkyl-guanidines described in the same patent as Cimetidine, i.e., U.S. Pat. No. 3,950,333.

In other embodiments, the selective H2 antagonist is Ranitidine (N[2-[[[5-{dimethylamino)methyl]-2-furanyl]methyl]thio]ethyl]-N′-methyl-2-nitro-1,1-ethene-diamine), which is described in U.S. Pat. No. 4,128,658. Ranitidine is commercialized as the hydrochloride salt under the brand name ZANTA™ among others, and is commonly used in the treatment of peptic ulcer disease, gastroesophageal reflux disease, and Zollinger-Ellison syndrome. Ranitidine bismuth citrate (sold under the trade name PYLORID™) is used to provide effective healing and symptom relief, both in duodenal ulcer disease and in gastric ulcer disease, and is often co-prescribed with certain antibiotics. Other H2 antagonists may be found among the aminoalkyl furan derivatives described in the same patent as Ranitidine, i.e., U.S. Pat. No. 4,128,658. Related compounds include AH-18801 (N-cyano-N′-(2-(((5-((dimethylamino)methyl)-2-furanyl)methyl)thio)ethyl)-N″-methyl-guanidine).

In still other embodiments, the selective H2 antagonist is Famotidine (YM-11170, MK-208, 3-[[2-(diaminomethylideneamino)-1,3-thiazol-4-yl]methylsulfanyl]-N′-sulfamoyl-propanimidamide), which is described in U.S. Pat. Nos. 4,283,408, 4,362,736. It is sold under the trade names PEPCID™ and PEPCIDINE™ among others, and is used to treat peptide ulcer disease, gastroesophageal reflux disease, and Zollinger-Ellison syndrome. Other H2 antagonists may be found among the guanidinothazole compounds described in the same patent as Famotidine, i.e., U.S. Pat. Nos. 4,283,408 and 4,362,736.

In other embodiments, the selective H2 antagonist is Nizatidine (LY-139037, ZL-101, N-[2-[[[2-[(dimethylamino)methyl]-4-thizaolyl]methyl]thio]ethyl]-N′-methyl-2-nitro-1,1-ethene-diamine), which is described in U.S. Pat. No. 4,375,547. Nizatidine is commercially available as the free base under the trade names TAZAC™ and AXID™ for the treatment of peptic ulcer disease and gastroesophageal reflux disease. Other H2 antagonists may be found among the N-alkyl-N′-((2-(aminoalkyl)4-thiazolymethyl)thioalkyl)guanidines, thioureas, ethenediamines and related compounds, which are described in the same patent as Nizatidine, i.e., U.S. Pat. No. 4,375,547.

In other embodiments, the selective H2 antagonist is Roxatidine (2-hydroxy-N-[3-[3-(1-piperidinylmethyl) phenoxy]propyl]acetamide acetate), which is described in U.S. Pat. No. 5,221,688. It is used to treat gastric ulcers, Zollinger-Ellison syndrom, erosive esophagitis, gastro-oesophageal reflux disease, and gastritis. It is commercialized as the acetate and it available in different countries, such as China, Japan, Korea, and South Africa. Other suitable H2 antagonists may be found among the phenoxypropylamine derivatives, which are described in the same patent as Roxatidine, i.e., U.S. Pat. No. 4,293,557.

In other embodiments, the selective H2 antagonist is Lafutidine ((Z)-2-[(2-furanylmethyl)sulfinyl]-N-[4-[[4-(1-piperidinyl-methy1)-2-pyridinyl]oxy]-2-butenyl]-acetamide), which is described in U.S. Pat. No. 4,912,101. It is marketed in Japan (STOGAR^{T}M) and in India (LAFAXID™), and is used to treat gastrointestinal disorders (gastric ulcers, duodenal ulcers, as well as wounds in the lining of the stomach associated with acute gastritis and acute exacerbation of chronic gastritis).

In other embodiments, the selective H2 antagonist is Niperotidine (N-(1,3-benzodioxol-5-ylmethyl)-N′-[2-[[[5-[(dimethylamino)methyl}-2-furanyl]methyl]thio]ethyl]-2-nitro-1,1-ethenediamine), which is described in U.S. Pat. No. 5,030,738.

In other embodiments, the selective H2 antagonist is Oxmetidine (SKF 92994, 5-(1,3-benzodioxol-5-ylmethyl)-2-[2-[(5-methyl-1H-imidazol-4-yl)methylsulfanyl]ethylamino]-1H-pyrimidin-6-one), which is described in EP Patent No. EP 01 126337.

In other embodiments, the selective H2 antagonist is Osutidine (T-593, (E)-N-[[[2-hydroxy-2-(4-hydroxyphenyl)ethyl]amino][[2-[[[5-[(methylamino)methyl]-2-furanyl]methyl]thio]ethyl]amino]methylenemethanesulphonamide), which is described in Japanese patents Nos. JP 09221422 and JP 03251527. Osutidine was developed by Toyama Chemical for the treatment of peptic/gastric and duodenal ulcers, but Toyama dropped it from clinical development in phase III trials in Japan.

In other embodiments, the selective H2 antagonist is Pibutidine (IT-066, 3-amino-4-{[(2Z)-4-{[4-(piperidin-1-ylmethyl)pyridin-2-yl]oxy}but-2-en-1-yl]amino}cyclobut-3-ene-1,2-dione), which is described in Japanese patents Nos. JP 05065226, JP 03251571 and JP 2858941. It was developped as the hydrochloride by Taisho Pharmaceutical for the treatment of duodenal ulcer and peptic ulcer, but was discontinued in phase III of clinical trials in Japan.

In other embodiments, the selective H2 antagonist is Lupitidine (SKF-93479, 2-((2-(((5-((dimethylamino)methyl)-2-uranyl)methyl)thio)ethyl)amino)-5-((6-methyl-3-pyridinyl)methyl)-4(1H)-pyrimidinone) or Donetidine (SKF-3574, 5-((1,2-dihydro-2-oxo-4-pyridinyl)methyl)-2-((2-(((5-(dimethylamino)methyl)-2-furanyl)methyl)thio) ethyl)amino)-4(1H)-pyrimidinone) or SKF-93828 (2-((2-(5-((4-(dimethylamino-methyl)-2-pyridyl)methyl)thio)ethyl)amino)-5-(2-methyl-5-pyridyl) pyrimidin-4-one), or SKF-93996 (the 2-(4-(4-(dimethylaminomethyl)-2-pyridyl) butylamino) analogue of SKF 93828), which are all described in U.S. Pat. No. 4,234,588.

In other embodiments, the selective H2 antagonist is Etintidine (BL-5641, BL-5641A, N-cyano-N′-(2-(((5-methyl-1H-imidazol-4-yl)methyl)thio)ethyl)-N″-2-propynyl-guanidine), which is described in U.S. Pat. No. 4,112,234. Other suitable H2 antagonists may be found among the imadazolylmethylthioethyl alkynyl guanidines, which are described in the same patent as Etintidine, i.e., U.S. Pat. No. 4,112,234.

In other embodiments, the selective H2 antagonist is Sufotidine (AH-25352, 1-methyl-3-((methylsulfonyl)methyl)-N-(3-(3-(1-piperidinylmethyl)phenoxy)propyl)-1H-1,2,4-triazol-5-amine), which is described in U.S. Pat. No. 4,670,448. It was developed as an antiulcerant by Glaxo, but its development was terminated from phase III clinical trials. Other suitable H2 antagonists may be found among the triazole amine compounds described in the same patent as Sufotidine (i.e., U.S. Pat. No. 4,670,448).

In other embodiments, the selective H2 antagonist is Tiotidine (ICI-125211, N-(2-(((2-((aminoiminomethyl)amino)A-thiazolyl)methyl)thio)ethyl)-N′-cyano-N″-methyl-guanidine), which is described in U.S. Pat. No. 4,165,378. Other suitable H2 antagonists may be found among the triazole amine compounds described in the same patent as Tiotidine (i.e., U.S. Pat. No. 4,165,378).

In other embodiments, the selective H2 antagonist is Ebrotidine (FI-3542, 4-bromo-N-[[[2-[[[2-[(diaminomethylene)amino]-4-thiazolyl]methyl]thio]ethyl]amino]methylene] benzenesulfonamide), which is described in U.S. Pat. No. 4,728,755. Ebrotidine is known to have gastroprotective activity against ethanol-, aspirin- or stress-induced gastric mucosal damage. The antisecretory properties of Ebrotidine are similar to those of Ranitidine, and approximately 10-fold greater than those of Cimeditine. Ebrotidine has been shown to be as effective as Ranitidine for the treatment of gastric or duodenal ulcers or erosive reflux oesophagitis. Other suitable H2 antagonists can be found among the sulfonamides described in the same patent as Ebrotidine (i.e., U.S. Pat. No. 4,728,755).

In other embodiments, the selective H2 antagonist is Bisfentidine (DA-5047, N-(1-methylethyl)-N′-(4-(2-methyl-1H-imidazol-4-yl)phenyl)-ethanimidamide), which is described in U.S. Pat. No. 4,649,150.

In other embodiments, the selective H2 antagonist is Dalcotidine (1-Ethyl-3-{3-[3-(1-piperidinylmethyl)phenoxy]propyl}urea), which was discontinued in phase III of clinical trials in Japan.

In other embodiments, the selective H2 antagonist is Impromidine (SKF 92676, 2-[3-(1H-imidazol-5-yl)propyl]-1-[2-[(5-methyl-1H-imidazol-4-yl)methylsulfanyl]ethyl]guanidine), which is described in UK Patent No. 1,531,237. Impromidine is a highly potent and specific histamine H2 receptor agonist that has been used diagnostically as a gastric secretion indicator. Other suitable H2 antagonists can be found among the N,N′-disubstituted guanidine compounds described in the same patent as Impromidine (i.e., UK Patent No. 1,531,237).

In other embodiments, the selective H2 antagonist is Lamtidine (AH-22216, 1-methyl-N5-(3-(3-1-piperidinylmethyl)phenoxy)propyl)-1H-1,2,4-triazole-3,5-diamine), which is described in U.S. Pat. No. 4,318,913. Alternatively, the H2 antagonist may be AH-21201 or AH-21272, which are also described in U.S. Pat. No. 4,318,913. Other suitable H2 antagonists can be found among the 1,2,4-triazole-3,5-diamine derivatives described in the same patent as Lamtidine (i.e., U.S. Pat. No. 4,318,913).

In other embodiments, the selective H2 antagonist is Lavoltidine (previously known as loxtidine, AH-23,844, 1-methyl-5-((3-(3-(1-piperidinylmethyl) phenoxy)propyl)amino)-1H-1,2,4-triazole-3-ethanol), which is described in U.S. Pat. No. 4,536,508. It is a highly potent and selective H2 receptor antagonist, which was under development by Glaxo Wellcome (now GlaxoSmithKline), for the treatment for gastroesophageal reflux disease but was discontinued due to the discovery that it produced gastric carcinoid tumors in rodents. Other suitable H2 antagonists can be found among the triazole amine derivatives described in the same patent as Lavoltidine (i.e., U.S. Pat. No. 4,536,508).

In other embodiments, the selective H2 antagonist is Mifentidine (DA-4577, N-(4-(1H-imidazol-4-yl)phenyl)-N′-(1-methylethyl)methanimidamide), which is described in U.S. Pat. No. 4,386,099. It is a highly potent and selective H2 receptor antagonist, which for the treatment for gastroesophageal reflux disease but was discontinued due to the discovery that it produced gastric carcinoid tumors in rodents. Other suitable H2 antagonists can be found among the imidazolylphenyl amidines described in the same patent as Mifentidine (i.e., U.S. Pat. No. 4,386,099).

In other embodiments, the selective H2 antagonist is Ramixotidine (CM-57755, N-(2-(((5-((dimethylamino)methyl)-2-furanyl)methyl)thio)ethyl)-3-pyridine carboxamide 1-oxide), which is described in U.S. Pat. No. 4,474,790. Other suitable H2 antagonists can be found among the thioalkylamide of nicotinic and 1-oxide compounds described in the same patent as Ramixotidine (i.e., U.S. Pat. No. 4,474,790).

In other embodiments, the selective H2 antagonist is Tuvatidine (HUK-978, 4-(((2-((5-amino-4-methyl-4H-1,2,4,6-thiatriazin-3-yl)amino)ethyl)thio)methyl)-2-thiazolyl) guanidine S,S-dioxide), which is described in Michel et al., “Synthesis of 4-Alkyl-1,2,4,6-thiatriazine 1,1-dioxide derivatives: Potent New Histamine H-2 Antagonists”, 190th ACS (Chicago), 1985, MEDI 33.

In other embodiments, the selective H2 antagonist is Zolantidine (SKF 95282, N-[3-[3-(1-piperidinylmethyl)phenoxy]propyl]-1,3-benzothiazol-2-amine). It is a brain-penetrating selective HRH2 antagonist developed by Smith, Kline & French. It is a benzothiazole derivative with a 30-fold higher potency for H2 receptors that other peripheral and central receptors.

In other embodiments, the selective H2 antagonist is BL-6341A (BMY-26539, 4-(((2-((4-amino-1,2,5-thiadiazol-3-yl)amino)ethyl)thio)methyl)-2-thiazolyl)-guanidine, S-oxide), which is described in U.S. Pat. No. 4,394,508, or one of the other 3-(hydroxy or amino)-4-(substituted amino)- and 3,4-di(substituted amino)-1,2,5-thiadiazole 1-oxides and 1,1-dioxides described in this U.S. patent.

In other embodiments, the selective H2 antagonist is BL-6548 (ORF-17910, N-(3-(3-((4-methyl-1-piperidinyl)methyl)phenoxy)propyl)-1,2,5-hiadiazole-3,4-diamine 1-oxide) or BMY-25271 (N-(2-(((5-(dimethylamino)methyl)-2-furanyl)methyl) thio)ethyl)-1,2,5-thiadiazole-3,4-diamine 1-oxide), which are described in U.S. Pat. No. 4,374,248, or one of the other 3-(hydroxy or amino)-4-substituted amino)- and 3,4-di(substituted amino)-1,2,5-thiadiazole-1-oxides and 1,1-dioxides described in this U.S. Patent.

In other embodiments, the selective H2 antagonist is BMY-25368 (SKF-94482, 3-amino-4-((3-(3-(1-piperidinylmethyl)phenoxy)propyl)amino)-3-cyclobutene-1,2-dione), which is described in U.S. Pat. No. 4,390,701, or one of the other 1-(substituted amino)-2-(amino or substituted amino)cyclobutene-3,4-diones disclosed in this U.S. patent.

In other embodiments, the selective H2 antagonist is BMY-25405 (N-(3-(3-(1-piperidinylmethyl)phenoxy)propyl)-1,2,5-thiadiazole-3,4-diaminemonohydro-chloride), which is described in U.S. Pat. Nos. 4,528,377, 4,600,779, or one of the other 3-(amino or substituted amino)-4-(substituted amino)-1,2,5-thiadiazoles described in these U.S. patents.

In other embodiments, the selective H2 antagonist is D-16637 (N-(2(((5-((tricyclo(2,2,1,0)hept-3-ylamino)methyl-2-furanyl)methyl)thio)ethyl)-N-methyl-2-nitro-1,1-ethenediamine hydrochloride), which is described in U.S. Pat. No. 4,738,983 or one of the other ethylenediamine and guanidine-derivatives disclosed in this U.S. Patent.

In other embodiments, the selective H2 antagonist is DA-4634 (4-(3-(((methylamino)methylene)amino)phenyl)-2-thiazolyl)-guanidine), which is described in U.S. Pat. Nos. 4,548,944 and 4,645,841, or one of the other guanidino-heterocyclyl-phenylamidines disclosed in these U.S. Patents.

In other embodiments, the selective H2 antagonist is FRG-8701 (N-(3-(3-(piperidinomethyl)phenoxy)propyl)-2-(furfurylsulfinyl)acetamide), which is described in U.S. Pat. No. 4,837,316, or one of the other alkylamide derivatives disclosed in this U.S. Patent.

In other embodiments, the selective H2 antagonist is FRG-8813 (N-(4-(4-(piperidinomethyl)pyridyl-2-oxy)-(Z)-2-butenyl)-2-(furfurylsulfiny-1)acetamide), which is described in U.S. Pat. Nos. 4,912,101 and 4,977,267, or one of the other 4-aminomethyl-pyridyl-2-oxy derivatives disclosed in these U.S. patents.

In other embodiments, the selective H2 antagonist is HB-408 (5-butyl-6-methyl-2-(3-(3-(piperidinomethyl) phenoxy)propylamino)pyrimidin-4(1H)-one), which is described in European Patent Application No. EP 0186275, or one of the other 2-substituted amino-4(1H)-pyrimidone derivatives disclosed in the EP patent application.

In other embodiments, the selective H2 antagonist is HE-30-256 (1-(3-(3-(piperidinomethyl)phenoxy)propylamino)-5-pyridin-2-sulfenamido-1,3,4-thiadiazole), which is described in U.S. Pat. No. 4,738,960, or one of the other 1,3,4-thiadiazole derivatives disclosed in the U.S. Patent.

In other embodiments, the selective H2 antagonist is L-64728 (4-amino-3-(2-(5-(dimethylaminomethyl)-2-furanymethylthio) ethylamino)-5-thoxycarbonylisothiazole-1,1-dioxide), which is described in European Patent Application No. EP 0040696 or one of the other 3,4-diamino-1,2,5-thiadiazole compounds disclosed in this EP patent application.

In other embodiments, the selective H2 antagonist is ICI-162846 (3-((imino((2,2,2-trifluoroethyl)amino)methyl)amino)-1H-pyrazole-1-pentanamide), which is described in U.S. Pat. No. 4,451,463, or one of the other alcohol guanidine derivatives disclosed in this U.S. patent.

In other embodiments, the selective H2 antagonist is ICIA-5165 (N-(4-(2-((aminoiminomethyl)amino)-4-thiazolyl)butyl)-N′-cyano-N″-methyl-guanidine), which is described in U.S. Pat. No. 4,165,377, or one of the other guanidine derivatives of imidazoles and thiazoles disclosed in this U.S. patent.

In other embodiments, the selective H2 antagonist is ORF-17578 (N-(2-(((5-((dimethylamino)methyl)-2-furanyl)methyl)thio)ethyl)-2-nitro-N′-2-propynyl,1-ethene diamine), which is described in U.S. Pat. No. 4,203,909, or one of the other N-alkynyl-N′-(omega-((5-substituted-2-furyl)alkylthio)alkyl)-derivatives of N″-cyanoguanidine and of 1,1-diamino-2-(substituted)-ethylene compounds disclosed in this U.S. patent.

In other embodiments, the selective H2 antagonist is SR-58042 ((N-(3-(3-(3-methyl)piperidinomethyl)phenoxy)propyl)-3-pyridinecarboxamide 1-oxide), which is described in U.S. Pat. No. 4,514,408, or one of the other N-substituted nicotin amide 1-oxide compounds disclosed in this U.S. patent.

In other embodiments, the selective H2 antagonist is Wy-45727 (N-(2-(((5-dimethylamino)methyl)-2-furanyl)methyl)thio)ethyl)thieno(3,4-d)isothiazol-3-amine 1,1-dioxide), which is described in U.S. Pat. No. 4,490,527, or one of the other benzo-fused heterocyclic compounds disclosed in this U.S. patent.

Other examples of suitable selective H2 antagonists include, but are not limited to, the compounds described in:
Borella et al., Arzneim. Forsch., 1988, 38(I): 366-372, such as AY-29315 (4-(dimethylamino)-N-(2-((4-((3-(3-(1-piperidinylmethyl)phenoxy)propyl)amino)-1,2,5-hiadiazol-3yl)amino)ethyl)butanamide S-oxide;
Muramatsu et al., Arzneim. Forsch., 1990, 40(I): 49-54, in particular IT-066 (3-imino-4-(4-(4-(1-piperidinomethyl)-2-pyridoxy)-cis-2-butenylamino)-3-cyclobutene-1,2-dione HCl);
Katz et al., J. Pharmacol. Experim.Therapeutics, 1987, 242: 437-442, in particular ORF-17583 (BL-6217, N-(2-(((5-((dimethylamino)methyl)-2-furanyl) methyl)thio)ethyl)-N′-methyl-1,2,5-thiadiazole-3,4-diamine 1-oxide);
Nielsen et al., Fed. Proc., 1984, Vol. 43, Abst. No. 4617, in particular Wy-45086 (N-(3-(3-((1-piperidinyl)methyl)phenoxy)propyl)-3-benzisothiazoleamine 1,1-dioxide) and Wy-45253 (N-(3-(3-(1-pyrrolidinyl-methyl)phenoxy)propyl)-1,2-benziso-thiazol-3-amine 1,1-dioxide HCl);
Tsuriya et al., Japan J. Pharmacol., 1984, 63(Suppl.): 90P-91P, in particular TAS (N,N-(3-(1-piperidinomethyl)phenoxy propyl)amino-5-amino-1,3,4-thiadiazole);
Oshita et al., Japan J. Pharmacol., 1986, 42: 229-235, in particular NO-794 (2-((3-(3-(1-piperidinylmethyl)phenoxy)propyl)amino-4(3H)-quinazolinone);
Nishida et al., Japan J. Pharmacol., 1991, 55 (Suppl 1) Abstract P497, in particular YM-14471 (2-(2-(2-diamino-methyleneamino)thiazol-4-ylmethylthio) ethyl)-5-(3-(diethylamino)propyl)-6-methyl-pyrimidin-4(1H)-one 3HCl);
Nelson et al., Agents and Actions, 1986, 19(3/4): 158-163, in particular Wy-45662 (N-(3-(3-(1-piperidinylmethyl)phenoxy)propyl)-thieno(3,4-d)isothiazol-3-ami ne 1,1-dioxide);
Hoffman et al., J. Med. Chem., 1983, 26: 140-144, in particular L-643441, N-(3-(3-(1-piperidinylmethyl)phenoxy)propyl)-1,2,5-thiadiazole-3,4-diamine 1-oxide; as well as:
FCE-23067 (2-guanidine-5-(N-isopropylcarbamoyl)-4,5,6,7-tetrahydrothiazole (5,4-c)pyridine), which is described in Arrigoni et al., Br. J. Pharmacol., 1985, 86: 780P; and
RGW-2568 (WHR-2568, N5-(3-((2,3-dihydro-1-(1-piperidinyl)-1H-inden-4-yl)oxy)propyl)-methyl-1H-1,2,4-triazole-3,5-diamine, which is described in Miksic et al., J. Chromatogr., 1988, 428: 113-121.

Other examples of suitable selective H2 antagonists include, but are not limited to:
5,6-substituted 4-pyrimidone compounds disclosed in Spengler et al., Agents and Actions, 1984, 14(3/4): 566-568;
3- and 2-indole derivatives disclosed in Tecle et al., J. Med. Chem., 1981, 24: 1095-1097;
benzylhistamine compounds disclosed in Emmett et al., J. Med. Chem., 1982, 25:1168-1174;
(imidazolylphenyl)guanidine, imidazolylbenzamidine, and (imidazolylphenyl) formamidine compounds disclosed in Donetti et al., J. Med. Chem., 1984, 27: 380-386;
N-cyano and N-carbamoyl amidine derivatives disclosed in Yanagisawa et al., J. Med. Chem., 1984, 27: 849-857;
biaryl pyridyl compounds disclosed in Lipinski et al., J. Med. Chem., 1985, 28(11): 1628-1636;
cimetidine analogs disclosed in Young et al., J. Med. Chem., 1986, 29: 44-49;
biaryl imidazolyl and triazolyl compounds disclosed in Lipinski et al., J. Med. Chem., 1986, 29: 2154-2163;
zwitterionic analogues of cimetidine disclosed in Young et al., J. Med. Chem., 1987, 30: 1150-1156;
N-substituted thieno(3,4-d)isothiazol-3-amine 1,1-dioxides and analogs disclosed in Santilli et al., J. Med. Chem., 1988, 31: 1479-1486;
pyrimidine and reduced pyrimidine analogues of Ranitidine disclosed in El-Badry et al., Eur. J. Med. Chem. Chim. Ther., 1985, 20(5): 403-407 and 409-413;
diaminofurazan compounds disclosed in Sorba et al., Eur. J. Med. Chem. -Chim. Ther., 1985, 20(6): 571-574;
Ranitidine analogues containing 5(6)substituted benzimidazole moieties disclosed in Sorba et al., Eur. J. Med. Chem.—Chim. Ther., 1986, 21(5): 391-395;
Cimetidine and Impromidine analogues disclosed in Sterk et al., Eur. J. Med. Chem., 1987, 22: 427-432;
pyridine and reduced pyridine analogues of cimetidine disclosed in El-Badry et al., Euro. J. Med. Chem., 1987, Vol. 22: 579-582;
N′-substituted thiourea, cyanoguanidine and dithiooxamide compounds disclosed in Barzen et al., Arch. Pharm. (Weinheim), 1981, 314: 617-622;
guanidinothiazole compounds disclosed in Trumm et al., Arzneim.-Forsch./Drug Res., 1983, 33(1)(2): 188-190, and Spengier et al., Arzneim-Forsch./Drug Res., 1983, Vol. 33(1)(3): 377-380;
N,N′-bisheteroaryl substituted cyanoguanidine and 2-nitro-1,1-ethenediamine compounds disclosed in Borchers et al., Arzneim.-Forsch./Drug Res., 1984, 34(II): 751-754;
1,2,5-thiadizole 1-oxide and 1,1-dioxide derivatives disclosed in Algieri et al., J. Med. Chem., 1982, 25(3): 210-212;
N-cyano and N-carbamoyl amidine derivatives disclosed in Yanagisawa et al., J. Med. Chem., 1984, 27(2): 849-857; and
Imidazo[1,2-a]pyridinylethylbenzoxazoles and related compounds disclosed in Katzura et al., Chem. Pharm. Bull (Tokyo), 1992, 40(6): 1424-1438.

Other selective H2 antagonists include, but are not limited to, the compounds described in:
U.S. Pat. No. 4,427,685, in particular N-(2-(((5-dimethylaminomethyl-2-furanyl)methyl)thio)ethyl)-N′-cyclo-octyl-2-nitro-1,1′-ethenediamine;
Borchers et al., Arzneim. Forsch., 1982, 32: 1509-1512, in particular N-cyano-N′,N″-bis(2-((5-methyl-4-imidazolyl)methylthio)ethyl) guanidine;
Elz et al., Arzneim.-Forsch., 1988, 38(I): 7-10, in particular N-cyano-N′-(2-(4,5,6,7-tetrahydrobenzimidazol-2-yl)ethyl)-1-N″-(2-((5-methylimidazol-4-yl)methyl thio)ethyl)guanidine;
Ueda et al., Chem. Pharm. Bull., 1990, Vol. 38(11): 3035-3041, in particular N-(3-(3-(1-piperidinylmethyl)phenoxy) propyl)-2-(2-hydroxyethylthio)acetamide;
Santilli et al., J. Med. Chem., 1988, 31: 1479-1486, in particular N-(3-(3-(1-piperidinyl)phenoxy)propyl)thieno(3,4-d)-isothiazol-3-amine-1,1-dioxide.

In other embodiments, the H2 antagonist is Icotidine (SK&F 93319, 2-[4-(3-methoxypyridin-2-yl)butylamino]-5-[(6-methylpyridin-3-yl)methyl]-1H-pyrimidin-6-one), which is a potent H1 and H2 antagonist.

In other embodiments, the selective H2 antagonist is Zaltidine (CP-57361, 2-[4-(2-methyl-1H-imidazol-5-yl)-1,3-thiazol-2-yl]guanidine), which is an effective but hepatotoxic H2-receptor antagonist (Farup, Scand. J. Gastroenterol., 1988, 23(3): 655-658).

H2 antagonists to be used in the context of the present invention (as candidate compounds and/or as therapeutic agents) may be prepared using conventional synthetic methods, or alternatively they can be obtained from commercial sources.

As indicated above, the present invention encompasses chemical derivatives of the above-listed H2 antagonists identified by one of the screening methods described herein as agents useful in the treatment or prevention of liver disease. Chemical derivatives are generally developed to exhibit increased biological efficacy.

As will be appreciated by one skilled in the art, H2 antagonists (including derivatives thereof) suitable for use in the present invention can be in a free from or in a pharmaceutically acceptable salt form. The term “pharmaceutically acceptable salt”, as used herein, refers to a salt that is, within the scope of sound medical judgment, suitable for use in contact with the tissues of subjects to be treated without undue toxicity, irritation, allergic response, and the like, commensurate with a reasonable benefit/risk ratio, and effective for their intended use.

Thus, the term “pharmaceutically acceptable salts” refers to the relatively non-toxic inorganic and organic acid addition or base addition salts of H2 antagonists (including of derivatives thereof). These salts can be prepared in situ during the final isolation and purification of the compounds or by separately reacting the purified compound in its free form with a suitable organic or inorganic acid or base and isolating the salt thus formed. Acid addition salts can be formed with inorganic acids (e.g., hydrochloric, hydrobromic, sulfuric, nitric, phosphoric acids, and the like) or organic acids (e.g., acetic, propionic, pyruvic, maleic, malonic, succinic, fumaric, tartaric, citric, benzoic, mandelic, methanesulfonic, ethanesulfonic, p-toluenesulfonic, salicylic acids, and the like). Base addition salts can be formed with inorganic bases (e.g., sodium, potassium, lithium, ammonium, calcium, magnesium, zinc, aluminum salts, and the like) or organic salts (e.g., salts of primary, secondary, and tertiary amines, substituted amines including naturally occurring substituted amines, cyclic amines, basic ion exchange resins, polyamine resins, and the like).

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in “Handbook of Pharmaceutical Salts: Properties, and Use” (2002).

It will be appreciated that many organic compounds can form complexes with solvents in which they are reacted or from which they are precipitated or crystallized. These complexes are known as “solvates”. Where the solvent is water, the complex is known as a “hydrate”. It will also be appreciated that many organic compounds can exist in more than one solid form, including crystalline and amorphous forms. All solid forms of the compounds described herein, including any derivatives thereof and solvates thereof, are within the scope of the present invention.

H2 antagonists suitable for use in the present invention may also exist in prodrug form. As used herein, the term “prodrug” refers to a pharmacologically inactive compound that is converted to an active drug by a metabolic biotransformation, which may occur prior, during and/or after absorption or at specific target sites within the body. Prodrug design may be useful in circumventing problems such as acid sensitivity, poor membrane permeability, drug toxicity, and short duration of action. Since prodrugs are known to enhance numerous desirable qualities of pharmaceuticals (e.g., solubility, bioavailability, manufacturing, etc.), the H2 antagonists employed in some methods of the invention may, if desired, be delivered in prodrug form. Thus, the invention contemplates prodrugs of H2 antagonists of the present invention as well as use thereof in methods of treatment or prevention of liver disease. Prodrugs of the H2 antagonists may be prepared by modifying functional groups present in the parent compound in such a way that the modifications are cleaved, either in routine manipulation or in vivo, to the parent compound. Accordingly, prodrugs include, for example, H2 antagonists described herein in which a hydroxy, amino, or carboxy group is bonded to any group that, when the prodrug is administered to a subject, cleaves to form a hydroxy, amino, or carboxylic acid, respectively. In particular, liver-targeted prodrugs of H2 antagonists may be prepared. The term “liver-targeted prodrug” refers to a pharmacologically inactive compound that is converted to an active drug by a metabolic biotransformation, which specifically occurs in the liver. Examples of methods for preparing liver-targeted prodrugs are known in the art (see, for example, Erion, “Prodrugs for Liver-Targeted Drug Delivery”, In: Stella et al. (eds.) “Prodrugs. Biotechnology: Pharmaceutical Aspects”, 2007, vol. V, Springer, New York, NY). They include, for example, a method for preparing liver-targeted prodrug derivatives of pharmacologically active molecules containing a phosphate or phosphonate group therein (U.S. Patent Publication No. US 2016/0115186); a method for preparing liver-targeted monophosphate prodrugs of alcohol-, amine- and thiol-containing drugs, known as cyclic 1-aryl-1,3-propanyl ester (HepDirect) prodrugs (Erion et al., J. Am. Chem. Soc., 2004, 126: 5154-5163; Erion et al., J. Pharmacol. Exp. Ther., 2005, 312: 554-560, U.S. Pat. Nos. 6,312,662 and 7,303,739).

In certain embodiments, although such may not be necessary, H2 antagonists identified by a screening method of the present invention, or chemical derivatives thereof, or (non-liver-targeted) prodrugs thereof, can optionally be targeted to the liver, using any known targeting means. The term “targeting to the liver” refers to the targeting of a compound or agent to a cell liver, such that at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, of the compound or agent administered to the subject enters the liver via the hepatic portal and becomes associated with (e.g., is taken up by) a cell liver.

Thus, the H2 antagonists described herein, the chemical derivatives thereof and the prodrugs thereof may be formulated with a wide variety of liver-targeted drug carriers. A used herein, the term “drug carrier” refers to a pharmaceutically acceptable substance formulated along with the active ingredient medication that is involved in carrying, delivering and/or transporting the active ingredient. The term “liver-targeted drug carrier” refers to a drug carrier that increases the effectiveness of drug delivery to the liver.

Examples of suitable drug carriers include, but are not limited to liposomes, microspheres (e.g., made of poly(lactic-co-glycolic) acid), albumin, albumin microspheres, lipoproteins, synthetic polymers, polymer conjugates, nanoparticles, nanofibers, protein-DNA complexes, protein conjugates, erythrocytes, virosomes, dendrimers, and recombinant chylomicrons, which are actively absorbed by the liver. More specific means include, but are not limited to, asialoglycopeptides (e.g. GalNAC); basic polyamino acids conjugated with galactose or lactose residues; galactosylated albumin; asialoglycoprotein-poly-L-lysine) conjugates; lactosaminated albumin; lactosylated albumin-poly-L-lysine conjugates; galactosylated poly-L-lysine; galactose-PEG-poly-L-lysine conjugates; lactosePEG-poly-L-lysine conjugates; asialofetuin; and lactosylated albumin.

Liver-targeted drug carriers have been described and reviewed (see for example, Nishikawa et al., J. Contr. Release, 1995, 36:1-2): 99-107; Fiume et al., Eur. J. Pharm. Sci., 2010, 40(4): 253-262; Tu et al., Curr. Top Med. Chem., 2013, 13(7): 857-866; Mishra et al., BioMed Research International, 2013, dx.doi.org/10.1155/2013/382184; Gorad et al., Int. J. Pharm. Sci. Res., 2013, 4(11): 4145-4157; WO/2013/176468; Wang et al., Current Drug Targets, 2014, 15: 573-599; Fiume et al., Expert Opin. Drug Deliv., 2014, 11(8): 1203-1217; U.S. Patent Publication No. US 2015/0297749; Singh et al., Biomaterials, 2016, 116: 130-144; Zhu et al., Acta Biomater., 2016, 30: 144-154; Cai et al., Mol. Pharm., 2016, 13(3): 669-709; Zhou et al., Anti-Cancer Agents in Med. Chem., 2017, 17(4): 1884-1897; Yan et al., Acta Biomater., 2017, 51: 363-373; Shamay et al., Nature Mater., 2018, 17: 361-368; Wu et al., J. Biomed. Nanotechnol., 2018, 14(11): 1837-1852; Wu et al., Front. Pharmacol., 2018, 9: 663; Chen et al., Eur. J. Med. Chem., 2019, 182: 111612).

III—Uses of the Identified H2 Antagonists, Derivatives Thereof and Prodrugs Thereof, in the Treatment and/or Prevention of Liver Disease, Including Hepato-Biliary Cancers

The present invention provides H2 antagonists identified by a screening method of the present invention, or chemical derivatives thereof, or prodrugs thereof (including liver-targeted prodrugs), for use in the treatment or prevention of liver disease in a subject. The present invention also relates to the use of such H2 antagonists, or chemical derivatives thereof, or prodrugs thereof, in the manufacture of a medicament for the treatment or prevention of liver disease in a subject. The present invention further relates to a method of treating or preventing liver disease in a subject, said method comprising a step of: administering to the subject in need thereof an effective amount of a H2 antagonist identified by a screening method of the present invention, or a chemical derivative thereof, or a prodrug thereof.

As used herein, the term “subject” refers to a human or another mammal (e.g., primate, dog, cat, goat, horse, pig, mouse, rat, rabbit, and the like), that can develop a liver disease, but may or may not be suffering from the disease. Non-human subjects may be transgenic or otherwise modified animals. In many embodiments of the present invention, the subject is a human being. In such embodiments, the subject is often referred to as an “individual” or a “patient”. The term “individual” does not denote a particular age, and thus encompasses newborns, children, teenagers, and adults. The term “patient” more specifically refers to an individual suffering from a disease. In the practice of the present invention, a patient will generally be diagnosed with a liver disease.

The term “treatment” is used herein to characterize a method or process that is aimed at (1) delaying or preventing the onset of a disease or condition (here a liver disease); (2) slowing down or stopping the progression, aggravation, or deterioration of the symptoms of the disease or condition; (3) bringing about amelioration of the symptoms of the disease or condition; or (4) curing the disease or condition. A treatment may be administered prior to the onset of the disease or condition, for a prophylactic or preventive action. Alternatively, or additionally, a treatment may be administered after initiation of the disease or condition, for a therapeutic action.

The terms “liver disease” and “hepatic disease” are used herein interchangeably and have their art understood meaning. They refer to any disturbance of liver function that causes illness. There are many different types of liver disease. Liver disease can be inherited (genetic) or caused by a variety of factors that damage the liver, such as viruses, alcohol use and obesity. A liver disease may be acute or chronic. Some of the most common types of liver disease include: acute liver failure, liver fibrosis, alcohol-related liver disease, fatty liver disease (including non-alcoholic fatty liver disease (NAFLD), such as non-alcoholic fatty liver (NAFL) and non-alcoholic steatohepatitis (NASH)), autoimmune liver disease (such as autoimmune hepatitis, primary biliary cholangitis, and primary biliary cirrhosis), cirrhosis (a chronic liver disease that causes advanced scarring of liver tissue), genetic liver diseases (such as hemochromatosis and Wilson disease), hepatitis (which is an inflammation of the liver that is most frequently due to viral infections, although it can also have other causes, such as exposure to chemicals, over-the-counter or prescription drugs, heavy alcohol use, inherited diseases, autoimmune disease, or fatty buildup in the liver) and hepato-biliary cancers (including hepatocarcinoma (HCC) and cholangial carcinoma (CC)).

The terms “hepatocellular carcinoma” and “HCC” are used herein interchangeably. They refer to the most common type of liver cancer, also called malignant hepatoma. HCC can be secondary to infection with hepatitis C virus (HCV), or secondary to hepatitis B virus (HBV) infection, alcoholic liver disease, non-alcoholic fatty liver disease, hereditary hemochromatosis, alpha 1-antitrypsin deficiency, auto-immune hepatitis, some porphyrias, Wilson's disease, aflatoxin exposure, type 2 diabetes, obesity, etc.

As used herein, the term “effective amount” refers to any amount of a compound, agent, antibody, or composition that is sufficient to fulfil its intended purpose(s), e.g., a desired biological or medicinal response in a cell, tissue, system or subject. For example, in certain embodiments of the present invention, the purpose(s) may be: to prevent the onset of a liver disease, to slow down, alleviate or stop the progression, aggravation or deterioration of the symptoms of the liver disease; to bring about amelioration of the symptoms of the disease, or to cure the disease.

### Administration

A H2 antagonist identified by a screening method described herein, or a derivative thereof, or a prodrug thereof, or a pharmaceutical composition thereof (see below), can be administered to a subject in need thereof using any suitable route. Various delivery systems are known and can be used, including tablets, capsules, injectable solutions, encapsulation in liposomes, microparticles, microcapsules, etc. Methods of administration include, but are not limited to, dermal, intradermal, intramuscular, intraperitoneal, intralesional, intravenous, subcutaneous, intranasal, pulmonary, epidural, ocular, and oral routes. A H2 antagonist, or a derivative thereof, or a prodrug thereof, or a pharmaceutical composition thereof, may be administered by any convenient or other appropriate route, for example, by infusion or bolus injection, by absorption through epithelial or mucocutaneous linings (e.g., oral, mucosa, rectal and intestinal mucosa, etc). Administration can be systemic or local. Parenteral administration may be preferentially directed to the patient's liver, such as by catheterization to hepatic arteries or into a bile duct. As will be appreciated by those of ordinary skill in the art, in embodiments where the H2 antagonist and additional biologically active agent(s) are administered sequentially (i.e., separately at different times or separately but at substantially the same time), the H2 antagonist and the additional biologically active agent(s) may be administered by the same route (e.g., intravenously) or by different routes (e.g., orally and intravenously).

### Dosage

Administration of a H2 antagonist identified by a screening method described herein, or a derivative thereof, or a prodrug thereof, or a pharmaceutical composition thereof, will be in a dosage such that the amount delivered is effective for the intended purpose. The route of administration, formulation and dosage administered will depend upon the therapeutic effect desired, the severity of the condition to be treated if already present, the presence of any infection, the age, sex, weight, and general health condition of the patient as well as upon the potency, bioavailability, and in vivo half-life of the liver disease therapeutic or chemopreventive agent used, the use (or not) of concomitant therapies, and other clinical factors. These factors are readily determinable by the attending physician in the course of the therapy. Alternatively, or additionally, the dosage to be administered can be determined from studies using animal models (e.g., chimpanzee or mice). Adjusting the dose to achieve maximal efficacy based on these or other methods are well known in the art and are within the capabilities of trained physicians.

A treatment according to the present invention may consist of a single dose or multiple doses. Thus, administration of a H2 antagonist identified by a screening method described herein, or a derivative thereof, or a prodrug thereof, or a pharmaceutical composition thereof, may be constant for a certain period of time or periodic and at specific intervals, e.g., hourly, daily, weekly (or at some other multiple day interval), monthly, yearly (e.g., in a time release form). Alternatively, the delivery may occur at multiple times during a given time period, e.g., two or more times per week; two or more times per month, and the like. The delivery may be continuous delivery for a period of time, e.g., intravenous delivery.

In general, the amount of a H2 antagonist identified by a screening method described herein, or a derivative thereof, or a prodrug thereof, or a pharmaceutical composition thereof, administered will preferably be in the range of about 1 ng/kg to about 500 mg/kg body weight of the subject, for example, between about 100 ng/kg and about 250 mg/kg body weight of the subject; or between about 1 μg/kg and about 100 mg/kg body weight of the subject, or between about 100 μg/kg and about 10 mg/kg body weight of the subject.

### IV—Pharmaceutical Compositions

A H2 antagonist identified by a screening method of the present invention, or a chemical derivative thereof, or a prodrug thereof (including a liver-targeted prodrug) may be incorporated into pharmaceutical compositions suitable for administration. Such pharmaceutical compositions comprise an effective amount of such H2 antagonist, or chemical derivative thereof, or prodrug thereof, and at least one pharmaceutically acceptable excipient. A pharmaceutical composition may further comprise one or more additional biologically active agents.

The term “pharmaceutically acceptable excipient” refers to a carrier medium which does not interfere with the effectiveness of the biological activity of the active ingredient(s) and which is not excessively toxic to the host at the concentration at which it is administered. The term includes solvents, dispersion media, coatings, antibacterial and antifungal agents, isotonic agents, adsorption delaying agents, and the like. The use of such media and agents for pharmaceutically active substances is well known in the art (see for example “Remington's Pharmaceutical Sciences”, E. W. Martin, 18^{th} Ed., 1990, Mack Publishing Co.: Easton, PA, which is incorporated herein by reference in its entirety).

A pharmaceutical composition according to the invention may be administered in any amount and using any route of administration effective for achieving the desired prophylactic and/or therapeutic effect. The optimal pharmaceutical formulation can be varied depending upon the route of administration and desired dosage. Such formulations may influence the physical state, stability, rate of in vivo release, and rate of in vivo clearance of the administered active ingredient.

The pharmaceutical compositions of the present invention may be formulated in dosage unit form for ease of administration and uniformity of dosage. The expression “unit dosage form”, as used herein, refers to a physically discrete unit of a compound identified by a screening method of the present invention as useful for the treatment or prevention of liver disease, or of a derivative thereof or of a prodrug thereof. It will be understood, however, that the total daily dosage of the pharmaceutical compositions will be decided by the attending physician within the scope of sound medical judgement.

### Formulation

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a non-toxic parenterally acceptable diluent or solvent, for example, as a solution in 2,3-butanediol. Among the acceptable vehicles and solvents that may be employed are water, Ringer's solution, U.S.P. and isotonic sodium chloride solution. In addition, sterile, fixed oils are conventionally employed as a solution or suspending medium. For this purpose, any bland fixed oil can be employed including synthetic mono- or di-glycerides. Fatty acids such as oleic acid may also be used in the preparation of injectable formulations. Sterile liquid carriers are useful in sterile liquid form compositions for parenteral administration.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Liquid pharmaceutical compositions which are sterile solutions or suspensions can be administered by, for example, intravenous, intramuscular, intraperitoneal or subcutaneous injection. Injection may be via single push or by gradual infusion. Where necessary or desired, the composition may include a local anesthetic to ease pain at the site of injection.

In order to prolong the effect of an active ingredient (i.e., a H2 antagonist identified by a screening method of the present invention, or a chemical derivative thereof, or a prodrug thereof), it may be desirable to slow the absorption of the ingredient from subcutaneous or intramuscular injection. Delaying absorption of a parenterally administered active ingredient may be accomplished by dissolving or suspending the ingredient in an oil vehicle. Injectable depot forms are made by forming micro-encapsulated matrices of the active ingredient in biodegradable polymers such as polylactide-polyglycolide. Depending upon the ratio of active ingredient to polymer and the nature of the particular polymer employed, the rate of ingredient release can be controlled. Examples of other biodegradable polymers include poly(orthoesters) and poly(anhydrides). Depot injectable formulations can also be prepared by entrapping the active ingredient in liposomes or microemulsions which are compatible with body tissues.

Liquid dosage forms for oral administration include, but are not limited to, pharmaceutically acceptable emulsions, microemulsions, solutions, suspensions, syrups, elixirs, and pressurized compositions. In addition to the active principles, the liquid dosage form may contain inert diluents commonly used in the art such as, for example, water or other solvent, solubilising agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cotton seed, ground nut, corn, germ, olive, castor, and sesame oils), glycerol, tetrahydrofurfuryl alcohol, polyethylene glycols, and fatty acid esters of sorbitan and mixtures thereof. Besides inert diluents, the oral compositions can also include adjuvants such as wetting agents, suspending agents, preservatives, sweetening, flavoring, and perfuming agents, thickening agents, colors, viscosity regulators, stabilizes or osmo-regulators. Examples of suitable liquid carriers for oral administration include water (potentially containing additives as above, e.g., cellulose derivatives, such as sodium carboxymethyl cellulose solution), alcohols (including monohydric alcohols and polyhydric alcohols such as glycols) and their derivatives, and oils (e.g., fractionated coconut oil and arachis oil). For pressurized compositions, the liquid carrier can be halogenated hydrocarbon or other pharmaceutically acceptable propellant.

Solid dosage forms for oral administration include, for example, capsules, tablets, pills, powders, and granules. In such solid dosage forms, an inventive combination may be mixed with at least one inert, physiologically acceptable excipient or carrier such as sodium citrate or dicalcium phosphate and one or more of: (a) fillers or extenders such as starches, lactose, sucrose, glucose, mannital, and silicic acid; (b) binders such as, for example, carboxymethylcellulose, alginates, gelatine, polyvinylpyrrolidone, sucrose, and acacia; (c) humectants such as glycerol; (d) disintegrating agents such as agar-agar, calcium carbonate, potato or tapioca starch, alginic acid, certain silicates, and sodium carbonate; (e) solution retarding agents such as paraffin; absorption accelerators such as quaternary ammonium compounds; (g) wetting agents such as, for example, cetyl alcohol and glycerol monostearate; (h) absorbents such as kaolin and bentonite clay; and (i) lubricants such as talc, calcium stearate, magnesium stearate, solid polyethylene glycols, sodium lauryl sulphate, and mixtures thereof. Other excipients suitable for solid formulations include surface modifying agents such as non-ionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. In the case of capsules, tablets and pills, the dosage form may also comprise buffering agents.

Solid compositions of a similar type may also be employed as fillers in soft and hard-filled gelatine capsules using such excipients as lactose or milk sugar as well as high molecular weight polyethylene glycols and the like. The solid dosage forms of tablets, capsules, pills, and granules can be prepared with coatings and shells such as enteric coatings, release controlling coatings and other coatings well known in the pharmaceutical formulating art. They may optionally contain opacifying agents and can also be of a composition such that they release the active ingredient(s) only, or preferably, in a certain part of the body (e.g., in the liver), optionally, in a delaying manner. Examples of embedding compositions which can be used include polymeric substances and waxes.

In certain embodiments, it may be desirable to administer a pharmaceutical composition locally to an area in need of treatment (e.g., the liver). This may be achieved, for example, and not by way of limitation, by local infusion during surgery (e.g., liver transplant), topical application, by injection, by means of a catheter, by means of suppository, or by means of a skin patch or stent or other implant.

For topical administration, the composition is preferably formulated as a gel, an ointment, a lotion, or a cream which can include carriers such as water, glycerol, alcohol, propylene glycol, fatty alcohols, triglycerides, fatty acid esters, or mineral oil. Other topical carriers include liquid petroleum, isopropyl palmitate, polyethylene glycol, ethanol (95%), polyoxyethylenemonolaurat (5%) in water, or sodium lauryl sulphate (5%) in water. Other materials such as antioxidants, humectants, viscosity stabilizers, and similar agents may be added as necessary.

Alternatively, a pharmaceutical composition may be disposed within transdermal devices placed upon, in, or under the skin. Such devices include patches, implants, and injections which release the active ingredient by either passive or active release mechanisms. Transdermal administrations include all administration across the surface of the body and the inner linings of bodily passage including epithelial and mucosal tissues. Such administrations may be carried out using the present compositions in lotions, creams, foams, patches, suspensions, solutions, and suppositories.

Transdermal administration may be accomplished through the use of a transdermal patch containing an active ingredient (i.e., a compound identified as useful for the treatment or prevention of liver disease by a screening method described herein or a derivative thereof) and a carrier that is non-toxic to the skin, and allows the delivery of the ingredient for systemic absorption into the bloodstream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may be suitable. A variety of occlusive devices may be used to release the active ingredient into the bloodstream such as a semi-permeable membrane covering a reservoir containing the active ingredient with or without a carrier, or a matrix containing the active ingredient.

Suppository formulations may be made from traditional materials, including cocoa butter, with or without the addition of waxes to alter the suppository's melting point, and glycerine. Water soluble suppository bases, such as polyethylene glycols of various molecular weights, may also be used.

Materials and methods for producing various formulations are known in the art and may be adapted for practicing the subject invention. Suitable formulations for the delivery of antibodies can be found, for example, in “Remington's Pharmaceutical Sciences”, E. W. Martin, 18^{th} Ed., 1990, Mack Publishing Co.: Easton, PA.

### Additional Biologically Active Agents

In certain embodiments, a H2 antagonist identified by a screening method of the present invention, or a chemical derivative thereof, or a prodrug thereof, is the only active ingredient in a pharmaceutical composition of the present invention. In other embodiments, the pharmaceutical composition further comprises one or more biologically active agents.

As used herein, the term “biologically active agent” refers to any molecule or compound whose presence in a pharmaceutical composition of the invention is beneficial to the subject receiving the composition. As will be acknowledged by one skilled in the art, biologically active agents suitable for use in the practice of the present invention may be found in a wide variety of families of bioactive molecules and compounds. Examples of suitable biologically active agents include, but are not limited to, therapeutic agents such as anti-viral agents, anti-inflammatory agents, immunosuppressive or immunomodulatory agents, analgesics, anti-apoptotic agents, antimicrobial agents, antibacterial agents, antibiotics, antioxidants, antiseptic agents, and combinations thereof.

In certain embodiments, the biologically active agent present in the pharmaceutical composition according to the present invention is selected among the drugs used in the treatment of liver disease. Examples of such drugs include, but are not limited to, corticosteroids, pentoxifylline, steroid-based drugs, antiviral drugs to treat viral hepatitis, anti-hypertensives, anti-diabetics, anti-inflammatory drugs, metabolism modifiers, other cancer agents, and the like.

In such pharmaceutical compositions, the H2 antagonist or a chemical derivative thereof or a prodrug thereof, and the one or more additional biologically active agent(s) may be combined in one or more preparations for simultaneous, separate or sequential administration of the different components. More specifically, a H2 antagonist, or a chemical derivative thereof or a prodrug thereof, may be formulated in such a way that the H2 antagonist, or chemical derivative thereof or prodrug thereof, and additional biologically active agent(s) can be administered together or independently from one another. For example, the H2 antagonist, or chemical derivative thereof or prodrug thereof, and an additional biological active agent can be formulated together in a single composition. Alternatively, they may be maintained (e.g., in different compositions and/or containers), for example in a kit, and then administered separately.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually performed, or data are actually obtained.

### Materials and Methods

HRH2 Blockers. Lafutidine (CAS Registry No. 118288-08-7), Cimetidine (CAS Registry No. 51481-61-9), Famotidine (CAS Registry No. 76824-35-6), Nizatidine (CAS Registry No. 76963-41-2 were purchased from Sigma; and Zaltidine (CAS Registry No. 85604-00-8) and Metiamide (CAS Registry No. 34839-70-8) were purchased from ChemScene; Roxatidine (CAS Registry No. 78273-80-0), and Ranitidine (CAS Registry No. 66357-35-5) were purchased from Ak Scientific; Etintidine (CAS Registry No. 69539-53-3), Oxmetidine (CAS Registry No. 72830-39-8), Lavoltidine (CAS Registry No. 76956-02-0), Sufotidine (CAS Registry No. 80343-63-1), Icotidine (CAS Registry No. 71351-79-6), Lupitidine (CAS Registry No. 83903-06-4), and Donetidine (CAS Registry No. 99248-32-5) were purchased from WuXi AppTec; Niperotidine (CAS Registry No. 84845-75-0) and Tiotidine (CAS Registry No. 69014-14-8) were purchased from Aqme.

Cells. Huh7.5.1 cells were cultured in Dulbecco's Modified Eagle Medium (DMEM) supplemented with 10% heat-decomplemented fetal bovine serum FBS, gentamycin (0.05 mg/mL) and non-essential amino acids (complete DMEM) at 37° C. with 5% CO₂. THP1 cells were cultured in RPMI 1640 Medium with GlutaMAX™-J supplement and HEPES and supplemented with 10% FBS and gentamycin (0.05 mg/mL). For proliferation arrest and differentiation (Huh7.5.1^{dif} cells), Huh7.5.1 cells were in complete DMEM supplemented with 1% DMSO (Choi et al., Biol., Syst. 2009, 39: 205-217; Sainz and Chisari, J. Virol., 2006, 80: 10253-10257). Primary human hepatocytes (PHH) were isolated from liver resection and cultured as previously described (Krieger et al., Hepatol., 2010, 51: 1144-1157). All cell lines were certified mycoplasma-free.

HCV Infection. Cell culture-derived HCVcc Jc1 (genotype 2a/2a) were produced in Huh7.5.1 cells as previously described (Pietschmann et al., PNAS USA, 2002, 103: 7408-7413; Wakita et al., Nature Med., 2005, 11: 791-796). HCV Jc1E2^{FLAG} was purified using anti-FLAG M2 affinity gel (Sigma-Aldrich) as described (Merz et al., J. Biol. Chem., 2011, 296: 3018-3032). HCVcc infectivity was determined by calculating the Tissue Culture Infective Dose 50 (TCID₅₀). To analyze the prognostic liver signature (PLS) induction, Huh7.5.1^{dif} cells were infected with HCV Jc1 or HCV Jc1E2^{FLAG}, for a total of 10 days. Cell culture supernatants from mock-electroporated cells or 100 μg/mL of FLAG peptide were used for control experiments. HCV infection was assessed by qRT-PCR of intracellular HCV RNA (Xio et al., PLOS Pathog., 2014, 10: e1004128) and immunostaining using HCV E2-specific AP33 antibody. To assess the effects of candidate compounds on the PLS, Huh7.5.1^{dif} cells were infected with HCV Jc1 for 7 days and treated with the different compounds for 3 additional days prior to cell lysis.

Histamine, 8-CPT, cAMP and H89 Treatment. Huh7.5.1^{dif} cells were cultured in DMEM containing 1% FBS and 1% DMSO and incubated with histamine (10 μM) or histamine+nizatidine (10 μM) for 48 hours. Fresh medium containing histamine was replenished every 12 hours. For 8-CPT cAMP and H89, Huh7.5.1^{dif} cells cultured in DMEM 1% DMSO and 1% FBS were incubated with 8-CPT cAMP (100 μM), H89 (1 μM) or DMSO as a control for 48 hours. Fresh medium containing 8-CPT cAMP was replenished every 12 hours.

CRISPR/Cas9 Gene Editing. (1) for Huh7.5.1: Lentiviruses expressing single guide RNA (sgRNA) were produced in HEK 293T cells by co-transfection with an envelope plasmid (pMD2.G), a packaging plasmid (psPAX2) and a lentiviral vector expressing the sgRNA (pXPR_BRD016) from the Broad Institute. Co-transfection was performed using the CalPhos™ Mammalian Transfection Kit (Clontech Laboratories) according to manufacturer's instructions. Huh7.5.1 stably expressing Cas-9 endonuclease (Huh7.5.1-Cas9) were generated by transduction of a lentiviral vector expressing Cas9 (pXPR_BRD111, Broad Institute). Huh7.5.1-Cas9 were DMSO differentiated for 7 days (Huh7.5.1-Cas9^{dif}) and were infected with HCV Jc1 for 7 days to induce the PLS as described above. Cells were then transduced with Lentiviruses expressing single guide RNA (sgRNA) CTRL targeting GFP (sgCTRL) or sgRNA targeting CREB5 (sgCREB5). After 48 hours, transduced cells were selected under hygromycin treatment (250 μg/ml) for 3 days prior cell lysis. The knock-out efficacy was assessed by Western blot analysis. For HRH2 KO, Huh7.5.1-Cas9 cells were then transduced with lentiviruses expressing single guide RNA (sgRNA) CTRL targeting GFP (sgCTRL) or sgRNA targeting HRH2 (sgHRH2) designed by the Broad Institute. After 48 hours, transduced cells were selected under hygromycin treatment (125 μg/ml). HRH2 KO was determined at genetic level using T7 endonuclease assay (Alt-R® Genome Editing Detection Kit, IDT™), according to manufacturer's instructions. (2) for THP1: HRH2 KO THP1 cells were engineered by Synthego (Synthego Corporation, Menlo Park, California, USA). Briefly, sgRNA targeting the beginning of HRH2 coding sequence were designed using Synthego CRISPR Design Tool (https://design.synthego.com) and complexed with S. pyogenes Cas9 2NLS nuclease to form ribonucleoproteins (RNPs) before cell transfection. Edited cells were then selected by clonal selection using Sony SH800 Cell Sorter (Sony, Serial number: 0314067).

Real-Time qRT-PCR. cDNAs were synthetized by reverse transcription using SuperScript III First-Strand Synthesis SuperMix (Life Technologies). Expression of mouse IL6, TNFα and IL1β was analyzed by quantitative real-time PCR using TaqMan Gene Expression Assays (Thermo Fisher Scientific) on the CFX96 Touch Real-Time PCR Detection System PCR system (Bio-Rad). Expression of human CCL2 and TGF was analyzed using iTaq™ Universal SYBR® Green Supermix (Bio-Rad). The 2^{-ΔCT} method was used for relative quantification (Schmittgen and Livak, Nature Protoc., 2008, 3: 1101-1108) of mRNA with normalization to 18S. The 2^{-ΔCT} method was used for relative quantification (Schmittgen and Livak, Nature Protoc., 2008, 3: 1101-1108) of mRNA with normalization to GAPDH mRNA.

Edu Assay and Flow Cytometry. HRH2 KO Huh7.5.1 cell proliferation was assessed using Click-iT® EdU Flow Cytometry Assay Kit (ThermoFischer Scientific) according to manufacturer's instructions. Briefly, HRH2 KO Huh7.5.1 or CTRL cells were incubated with EdU (5-ethynyl-2′-deoxyuridine) for 3 hours at 37° C. Then, celles were detached and 200,000 cells per condition were fixed with paraformaldehyde 0.5% and permeabilized using 0.2% saponin+1% FBS before being incubated with Click-iT® EdU detection cocktail Edu incorporation and analyzed by Flow Cytometry. Data were acquired using Cytoflex B2R2V0 (Beckman Coulter, BA47394) and CytExpert 2.3 software and then analyzed using FlowJo V10.5.1. Celles were incubated without Edu but expoxed to the detection cocktail used as reference. For PHH, HRH2 was stained using rabbit polyclonal HRH2-specific antibody. Cytokeratin 18 (CK18) was used as hepatocyte marker (mouse monoclonal anti-CK18 antibody). Rabbit or mouse control IgGs were used as negative controls. Primary antibodies were detected using Alexa Fluor™ 647-conjugated rabbit-specific or PE-conjugated mouse-specific secondary antibodies according to manufacturer's instructions. Data were acquired using Sony SH800 Cell Sorter (Sony, 0314067) and SH800 cell sorter software V2.1.5 then analyzed using FlowJo V10.5.3.

ELISA Assay. To assess the HCV infection and nizatidine treatment on intracellular cAMP level, Huh7.5.1^{dif} cells were infected with HCV Jc1 for 5 days and were incubated for 3 more days with nizatidine or DMSO as a control. The cAMP level was measured by ELISA assay using the cAMP Parameter Assay Kit (R&D System) according to manufacturer's instructions.

Patient-Derived HCC Tumor Spheroids. Tumorspheroids were generated from liver tissues from HCC patients undergoing surgical resection and dissociated using the Human Tumor Dissociation Kit. Total cell populations including epithelical (i.e., cancer cells/hepatocytes) and NPCs were used to generate multicellular tumorspheroids in Corning® 96-well Black/Clear Bottom Low Flange Ultra-Low Attachment Microplates (Corning). After 48 hours, HCC-derived spheroids were treated with nizatidine 50 μM, sorafenib 10 μM or DMSO vehicle control for 4 days. Fresh medium containing DMSO or drugs was added every day. Cell viability was assessed using CellTiter-Glo® (Luminescent Cell Viability Assay), according to manufacturer's instruction.

NPCs was used to generated multicellular tumorspheroids in Corning® 96-well Black/Clear Bottom Low Flange Ultra-Low Attachment Microplate (Corning).

Single-Cell RNA-Seq Analyses of Patient Liver Tissues. Human cells were dissociated from HCC patient liver tissues. Fresh or cryopreserved tissues were minced into 2 to 3 mm pieces and incubated with collagenase (0.5 mg/mL) for 30 minutes at 37° C. Digested tissues were filtered through a sterile nylon mesh to separate the dispersed cells from tissue fragments and washed with Hanks' Balanced Salt Solution (HBSS). Cells were then centrifuged at 600×g for 5 minutes at 4° C. Leucocytes CD45⁺ cells were separated from other cell types by flow cytometry using SH800 cell sorter (Sony). Briefly, 500,000 cells per condition were stained with human CD45 specific antibody. Living cells were selected using Zombie green staining according to manufacturer's instructions. Data were acquired using the Sony Cell Sorter Software. Cells were then cultured in MammoCult™ medium and treated with nizatidine 50 μM or DMSO vehicle control. Two days after treatment, single cells were sorted into 384 well cell capture plates (Single Cell Discovery, website: www.scdiscoveries.com) using SH800 cell sorter (Sony). The plates contain mineral oil and droplets of poly-A primers, containing a cell-specific barcode and a unique molecular identifier (UMI), enabling to distinguish the well-specific (and cell-specific) mRNA molecules. scRNA-Seq was performed by Single-Cell Discoveries B.V. using SORT-seq protocol (Muraro et al., Pancreas Cell Syst., 2016, 3: 385-394, e3).

M1 Macrophage Polarization and HRH2 Blocker Treatment. THP1 cells were cultured in RPMI 1640 Medium with GlutaMAX™-I supplement and HEPES and supplemented with 10% FBS and gentamycin (0.05 mg/mL). M1 macrophage polarization from THP-1 was performed as previously described (Yeung et al., J. Hepatol., 2015, 62: 607-616). To generate MO THP-1 macrophages, THP1 cells were treated with PMA 320 nM for 24 hours. To generate M1-polarized THP-1 macrophages, THP-1 cells were treated with 320 nM for 24 hours and then with 100 ng/ml LPS and 20 ng/ml IFNγ for 18 hours. Cells then were treated with HRH2 blockers 20 μM or DMSO vehicle for 72 hours.

RNA-Seq of M1 macrophages. RNA-Seq was performed on total RNA from MO macrophages, M1-polarized macrophages, and M1-polarized macrophages treated with Oxmetidine, Nizatidine or DMSO (triplicates). RNA-Seq was performed by the Biomedical Sequencing Facility (BSF) CEMM Research Center for Molecular Medicine of the Austrian Academy of Sciences, Vienna (Austria). Briefly, RNA-Seq libraries were generated from 300 ng of total RNA using TruSeq Stranded mRNA Sample Preparation Kit (Illumina, Part Number RS-122-2101). Following purification with poly-T oligo attached magnetic beads, the mRNA was fragmented using divalent cations at 94° C. for 2 minutes. The cleaved RNA fragments were copied into first strand cDNA using reverse transcriptase and random primers. Strand specificity was achieved by replacing dTTP with dUTP during second strand cDNA synthesis using DNA Polymerase I and RNase H. Following addition of a single ‘A’ base and subsequent ligation of the adapter on double stranded cDNA fragments, the products were purified and enriched with PCR (30 seconds at 98° C.; [10 seconds at 98° C., 30 seconds at 60° C., 30 seconds at 72° C.]×12 cycles; 5 minutes at 72° C.) to create the cDNA library. Surplus PCR primers were further removed by purification using AMPure XP beads (Beckman Coulter) and the final cDNA libraries were checked for quality and quantified using 2100 Bioanalyzer (Agilent). Libraries were sequenced on the Illumina HiSeq 4000 as Single-Read 50 base reads following Illumina's instructions. Image analysis and base calling were performed using RTA v2.7.3 and bcl2fastq v2.17.1.14.

Isolation and Treatment of Patient-Derived Kupffer Cells. Kupffer cells were isolated as previously described (Kegel et al., J. Vis. Exp., 2016, 109: e53069) from liver tissue of patients without history of chronic liver disease. Briefly, the non-parenchymal cell populations (NPCs) were purified by serial centrifugation followed by separation of Kupffer cells, exploiting fast attachment of primary macrophages to plastic within 20 minutes. Cells were cultured in RPMI 1640 Medium with GlutaMAX™-I supplement and HEPES and supplemented with 10% FBS and gentamycin (0.05 mg/mL). To generate M1 macrophages, Kupffer cells were treated with 100 ng/ml LPS and 20 ng/ml IFNγ for 18 hours. Cells then were treated with nizatidine 30 μM or DMSO vehicle for 48 hours before assessing cytokine expression by qRT-PCR.

Transcriptomic Analysis and PLS Calculation. Gene expression profiling and the 32 gene PLS 32 profiling were performed using 250-500 ng total RNA by using either nCounter Digital Analyzer system (NanoString) or the HumanHT-12 beadarray (Illumina) for the time-course experiments. PLS gene expression was normalized according to 6 housekeeping gene expression using GenePattern genomic analysis toolkits (Hoshida, PLOS ONE, 2010, 5: e15543; Peck et al., Genome Biol., 2006, 7: R61; Reich et al., Nature Genet., 2006, 38: 500-501). Induction or suppression of PLS risk signature was determined as previously reported by using Gene Set Enrichment Analysis (GSEA), implemented in GenePattern genomic analysis toolkits (Hoshida, PLOS ONE, 2010, 5: e15543; Peck et al., Genome Biol., 2006, 7: R61; Reich et al., Nature Genet., 2006, 38: 500-501). PLS was always determined by using CTRL cells as references. Results are presented as simplified heatmaps showing the classification of PLS global status as poor or good prognosis and the significance of induction/suppression of PLS genes (log 10 of false discovery rate (FDR) values). For each experiment, heatmaps show: in the upper part: the classification of PLS global status as poor (orange) or good (green) prognosis, and in the bottom part: the significance of induction (red) or suppression (blue) of PLS poor- or good-prognosis genes. Global status corresponds to the difference between low risk- and high risk-gene expression. The statistic tests, normalized enrichment score (NES) and FDR values are provided for each PLS experiment (poor- and good-prognosis genes variation and global status) in the Source Data file.

Single Cell RNA-Seq Profiling of Human Cells. scRNA-Seq analysis of patient-derived liver tissue was assessed using R version 3.5.3 with package “RaceID” for clusterization, cluster analysis and DEG calculation, as previously described (Aizarani et al., Nature; 2019, 572: 199-204).

In vitro Apoptosis Assay. Apoptosis level in HRH2 KO or CTRL Huh7.5.1 cells was assessed by detecting cleaved caspase 3 after H₂O₂ treatment (300 PM, 3 hours) using CellEvent™ Caspase-3/7 (ThermoFischer Scientific), according to manufacturer's instructions. Immunofluorescence pictures were acquired using Axio Observer Z1 microscope. Quantification of cleaved caspase 3 levels were performed using Celigo Imaging Cytometer. Data were integrated and normalized to total cell number (DAPI staining).

Data and Software Availability. All genomic datasets used for this study are available at NCBI Gene Expression Omnibus database (website: ncbi.nlm.nih.gov/geo, accession number: GSE66843).

Transability. For perturbation studies in the cell-based system, the dose of nizatidine was chosen according to the nizatidine blood concentrations measured in patients. Plasma concentrations reach between 700 and 1400 μg/ml (2-4 μM) after a 300 mg oral dose.

Statistics. In vitro data are presented as the mean±s.d. and were analyzed by the unpaired Student's t-test or the two-tailed Mann-Whitney test as indicated after determination of distribution by the Shapiro-Wilk normality test and homoscedasticity tests. All in vitro experiments were performed at least in triplicates and repeated 2 or 3 times and considered as significant at p<0.05. Statistical analyzes for in vitro experiments were performed with GraphPad Prism 6 software.

### Example 1: Fast-Track Liver Disease Chemoprevention Discovery Using a Clinical Gene-Signature-Inducible Human Cell Culture Model—Drug Discovery Targeting a Prognostic Liver Signature Uncovers HRH2 Antagonists for the Treatment of Liver Fibrosis and Cancer Prevention

### Results

Inducible Liver Cell-Based System Recapitulating the Cellular PLS in Cell Culture, Infection with Hepatitis Viruses and Metabolic Injuries in Cell-Based Models Induce the PLS Similar to Clinical Cohorts. The prognostic liver signature (PLS) cell model developed by the present inventors, and used in the studies presented herein, has previously been described (WO 2016/174130). Overall, the cPLS model offers unique opportunities to discover compounds for chronic liver disease treatment and HCC chemoprevention across the distinct liver cancer etiologies, in a fast-track high-throughput screening format. The innovation of the cPLS model compared to other 2D and 3D model systems for liver disease is its read-out of a clinically relevant PLS predicting disease progression and HCC risk, which enables a novel approach of drug and target discovery not provided by other models. Of note, the cPLS system is amenable to genetic perturbation as well, such as CRISPR-Cas9 gene editing.

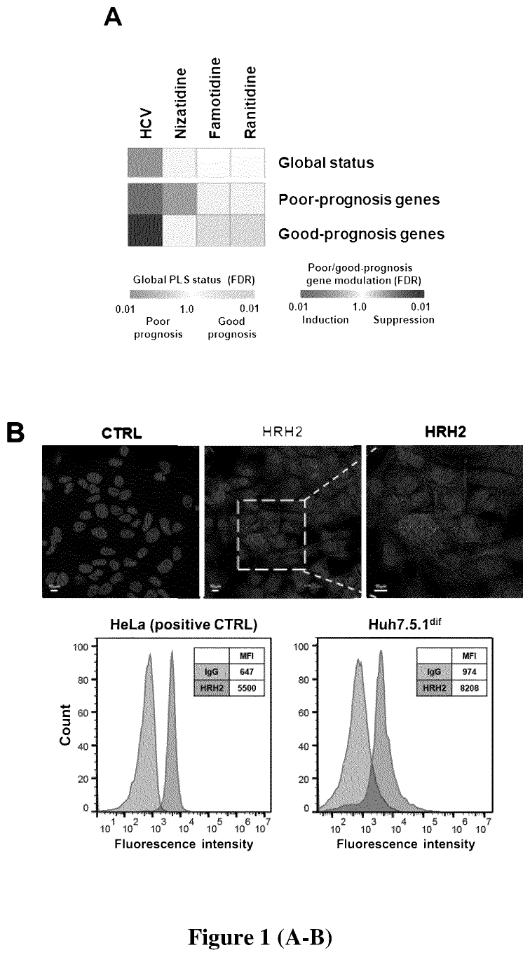

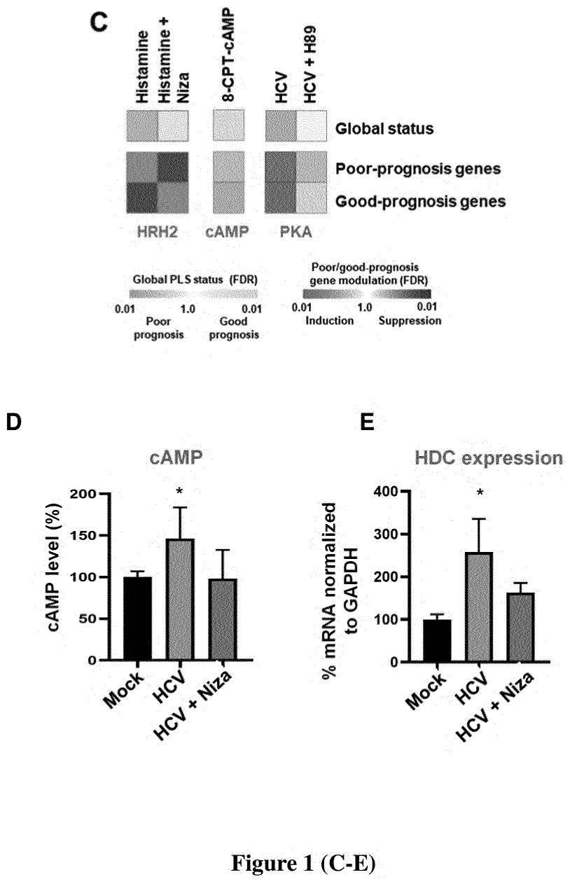

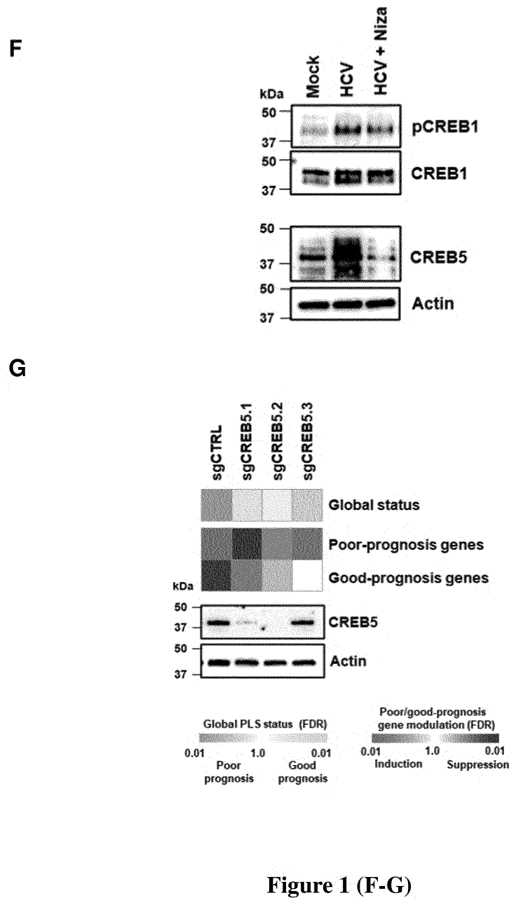

Nizatidine Reverses the Poor-Prognosis Status of the Cellular PLS by Inhibiting the HRH2/cAMP CREB Signaling Pathways. Screening of computationally prioritized compounds in the cPLS models identifies Nizatidine, a histamine receptor H2 (HRH2) antagonist, as the PLS-reversing compounds with highest statistical significance (FDR<0.05). Of note, other HRH2 blockers (e.g., Famotidine, Ranitidine) also showed a partial reversal of the PLS in the cPLS model, suggesting a class-effect of HRH2 inhibitors on the PLS (FIG. 1A).

HRH2 is a member of the G protein-coupled receptor family widely expressed in the gastrointestinal tract that mediates its activity through cAMP and PKA (Unen et al., Mol. Pharmacol., 2016, 90: 162-176). To investigate the mechanism of action of Nizatidine in the developed cell-based system, the expression of HRH2 in Huh7.5.1^{dif} cells was confirmed by flow cytometry and immunofluorescence analyses (FIG. 1B). Induction of the PLS poor-prognosis status by histamine, the natural ligand of HRH2, with reversal by Nizatidine, demonstrated the functional activity of HRH2 in Huh7.5.1^{dif} cells and confirmed the functional impact of HRH2 in the modulation of the PLS (FIG. 1C). These results were corroborated by an increase in intracellular cAMP level following induction of the poor-prognosis PLS, reverted after Nizatidine treatment (FIG. 1D). Furthermore, incubation of the cells with 8-CPT-cAMP, a cAMP analogue, resulted in induction of the poor-prognosis status of the PLS (FIG. 1C). In addition, treatment of the cells with H89, a specific PKA inhibitor, reversed the induction of the PLS poor-prognosis status, demonstrating the involvement of the cAMP/PKA axis in the induction of the PLS (FIG. 1C). Of note, Huh7.5.1^{dif} cells produce low but detectable levels of histamine ranging from 1.8 to 3.1 ng/mL in cell culture supernatant and express histidine decarboxylase (HDC), the enzyme catalyzing histamine production from histidine (FIG. 1E). Interestingly, HDC expression increased upon HCV infection, suggesting that these cells produce more histamine for stimulation of histamine receptors in stress conditions (FIG. 1E). These data are in line with different studies showing that malignant cells, including hepatoma cells, express histamine receptors and secrete histamine in micromolar concentrations in the extracellular medium (Francis et al., Gut, 2012, 61: 753-764; Kennedy et al., Transl. Gastrointest. Cancer, 2012, 1: 215-227; Lampiasi et al., Exp. Mol. Med., 2007, 39: 284-294).

Increase in intracellular cAMP is known to activate the cAMP response element binding (CREB) protein family, which has key functions in different cellular processes including cell survival, growth and differentiation (Steven et al., Oncotarget, 2016, 7: 35454-35465). The present Inventors decided to focus on CREB1 and CREB5, two key members of the CREB family found to be overexpressed in many solid tumors including HCC (Abramovitch et al., Cancer Res., 2004, 64: 1338-1346; He et al., Oncol., Lett., 2017, 14: 8156-8161; Steven et al., Oncotarget, 2016, 7: 35454-35465). Interestingly, induction of the PLS poor-prognosis status by persistent HCV infection resulted in an increase of CREB1 phosphorylation and of CREB5 expression. Importantly, both CREB1 phosphorylation and CREB5 overexpression were reversed by Nizatidine (FIG. 1F), suggesting a role of these transcription factors in the induction of the PLS poor prognosis status. Indeed, loss-of-function studies using CRISPR/Cas9 revealed that knock-out of CREB5 reversed the induction of the PLS poor-prognosis status, confirming CREB5 as a key driver of the PLS (FIG. 1G). Collectively, the results obtained demonstrate that the HRH2/cAMP/CREB signaling pathway is a driver of the PLS in the cell-based system and plays a functional role in HCC growth.

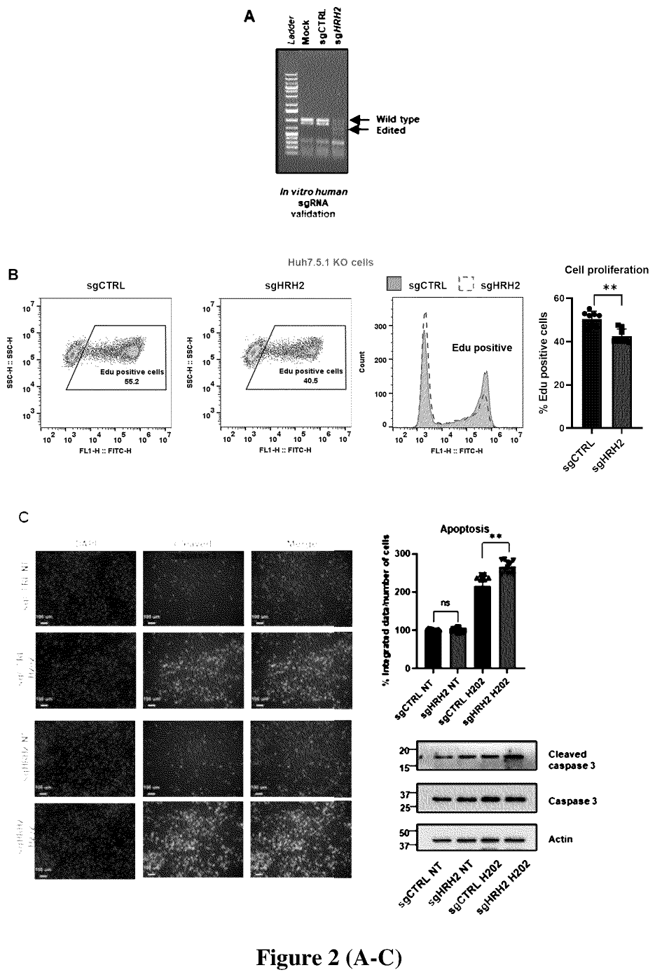

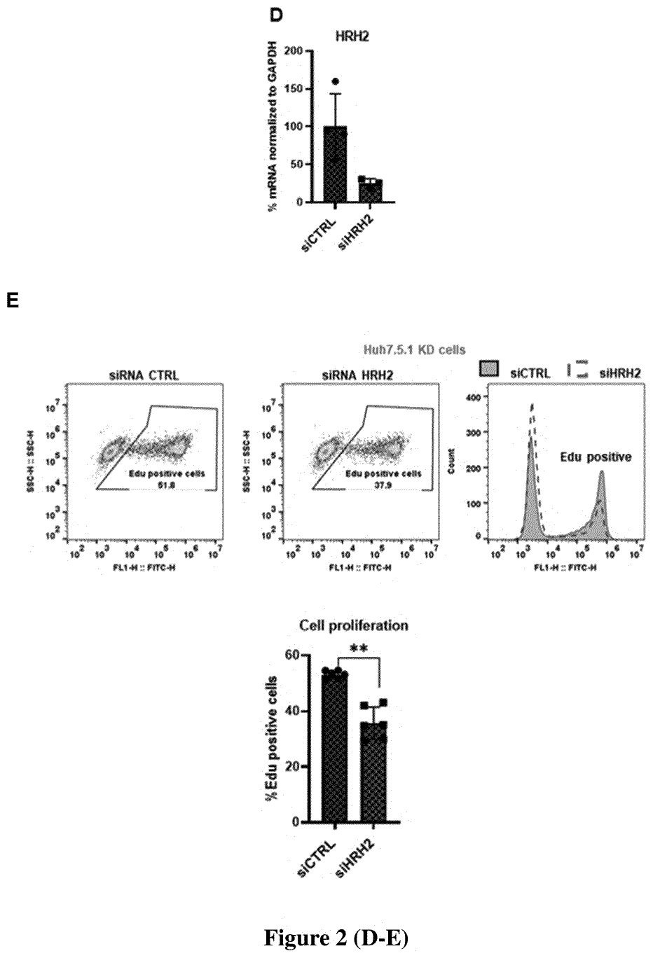

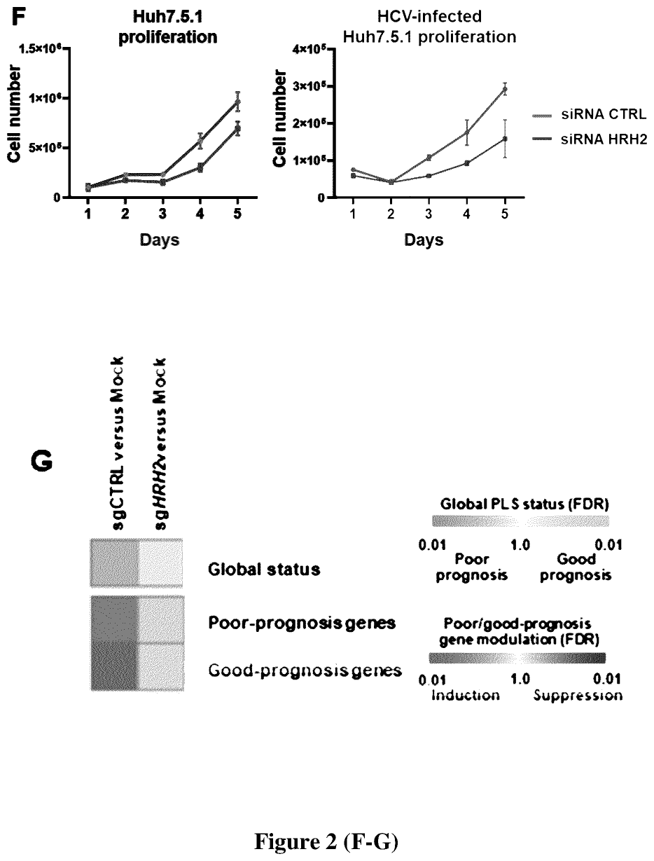

Genetic Loss-of-function Studies Confirm a Functional Role of HRH2 in Hepatocarcinogenesis. To investigate the functional role of HRH2 on the phenotype of hepatoma cells, the inventors engineered Huh7.5.1 HRH2 KO and control KO cell lines. HRH2 KO hepatoma cells showed decreased cell proliferation (FIGS. 2A-B) and increased sensitivity to oxidative stress and apoptosis compared to cells expressing a sgCTRL (FIG. 2C). A decrease in Huh7.5.1 cell proliferation was also observed when HRH2 expression was knocked-down by RNAi (FIGS. 2D-F). Moreover, HRH2 KO impaired the full induction of the poor-prognosis status of the PLS compared to CTRL cells (FIG. 2G). Collectively, these genetic loss-of-function studies suggest that HRH2 plays a functional role in hepatocarcinogenesis and that the biological effects of Nizatidine on liver disease progression are likely mediated by HRH2. Nevertheless, our data do not exclude that additional mechanisms or targets are at play.

Nizatidine Improves Liver Disease and Prevents Cancer by Targeting HRH2⁺, CLEC5A^{high}, MARCOO^{low} Liver Macrophages. Increasing evidence has shown that the interplay between hepatocytes and the surrounding microenvironment plays an important role in liver disease progression and hepatocarcinogenesis. In particular, the recruitment of inflammatory immune cells in the chronically injured tissue is crucial for driving fibrosis and HCC (Amicone et al., Transl. Gastroenterol. Hepatol., 2018, 3: 24). Given the role of histamine and histamine receptors in the regulation of immune responses and inflammation, i.e., in inflammatory lung diseases (O'Mahony et al., J. Allergy Clin. Immunol., 2011, 128: 1153-1162), the Inventors hypothesized that targeting HRH2 using Nizatidine may modulate liver inflammation and immunity and therefore improve liver disease and carcinogenesis.

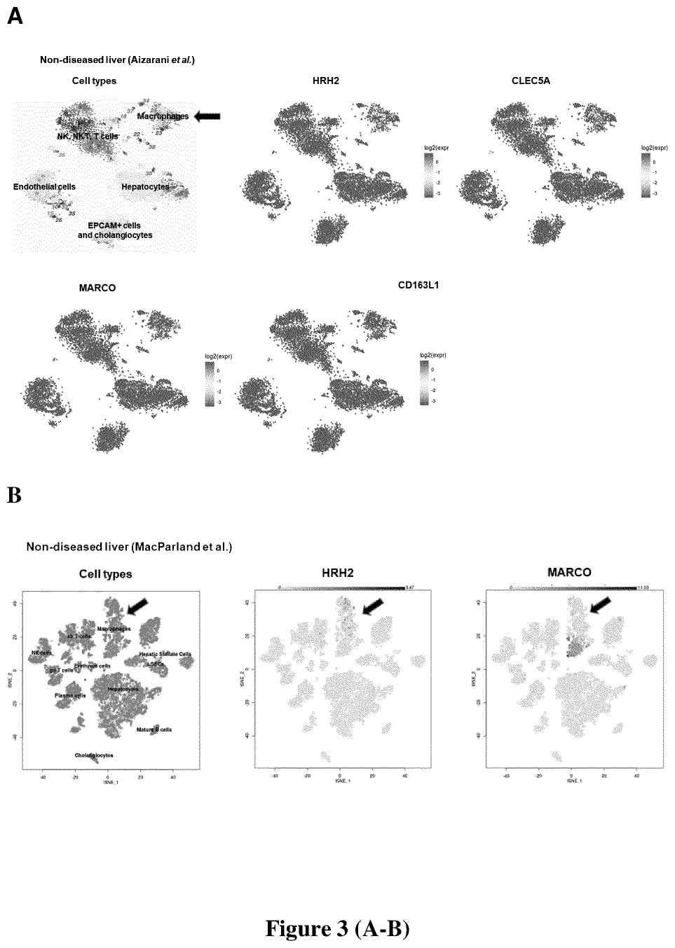

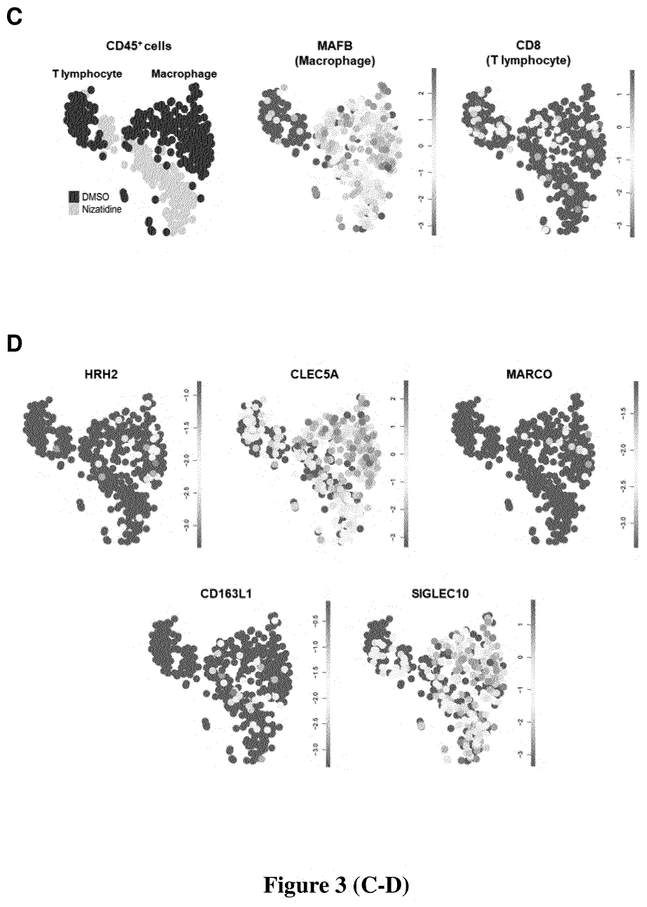

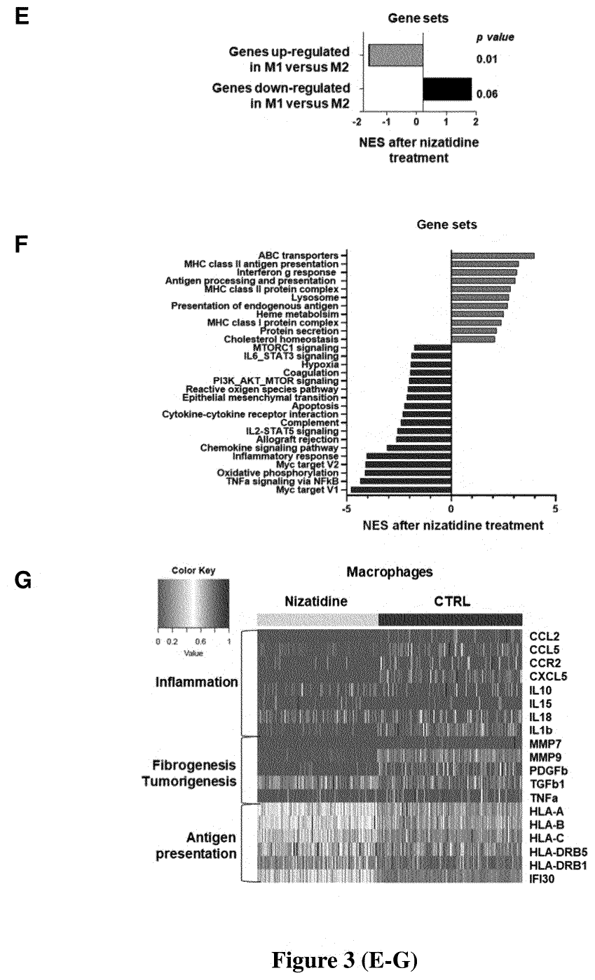

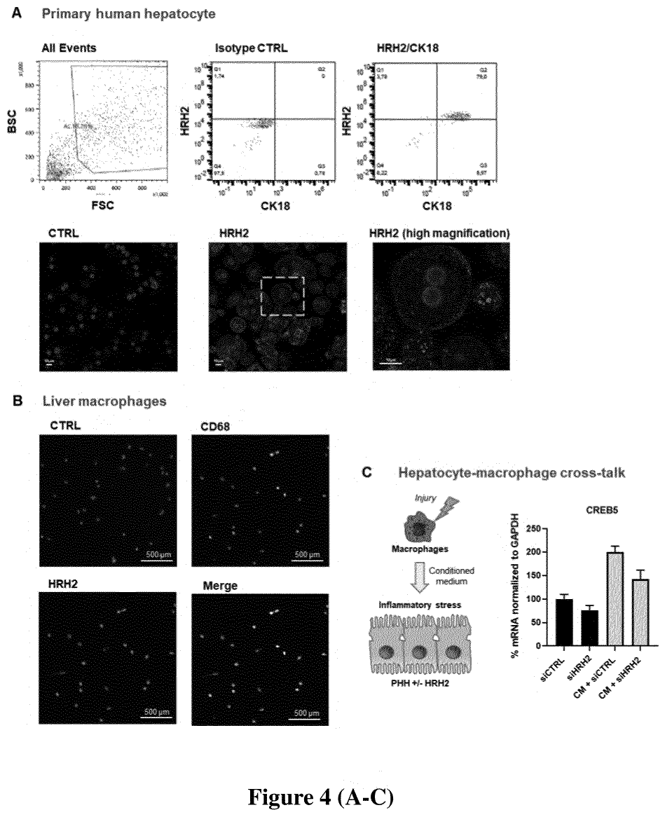

To uncover the immune cells targeted specifically by Nizatidine, the Inventors first analyzed target expression in the healthy human liver using scRNA-Seq. ScRNA-Seq of human liver cells from non-diseased livers of the human liver cell atlas (Aizarani et al., Nature, 2019, 572: 199-204; MacParland et al., Nature Commun., 2018, 9: 4383) showed that HRH2 is highly expressed in liver macrophages (FIG. 3A-B). Immunofluorescence analysis performed on isolated macrophages and hepatocytes confirmed that HRH2 is expressed in macrophages as well as hepatocytes with higher levels in macrophages (FIG. 4). Interestingly, HRH2-expressing macrophages express high levels of C-type lectin domain family 5 member A (CLEC5A), which is involved in signaling transduction and production of pro-inflammatory cytokines (Gonzilez-Dominguez et al., J. Leukoc. Biol., 2015, 98: 453-466) and low levels of the immunoregulatory macrophages expressing the Macrophage Receptor with Collagenous structure (MARCO) marker (Gonzilez-Dominguez et al., J. Leukoc. Biol., 2015, 98: 453-466; MacParland et al., Nature Commun., 2018, 9: 4383). HRH2 is mainly expressed in CLEC5A^{high}, MARCO^{low} cells, suggesting that pro-inflammatory macrophages are most likely Nizatidine targets in patients (FIG. 3A-B).

To investigate the functional effects of Nizatidine treatment on liver immune cells, the present inventors isolated CD45⁺ leucocyte cells from liver tissue of patients with advanced liver disease and HCC and treated the immune cell population with Nizatidine or vehicle control. They then analyzed the effect of the compound by scRNA-Seq using the SORT-seq technology (Muraro et al., Cell Syst., 2016, 3: 385-394.e3) similar to their previously described liver cell atlas pipeline (Aizarani et al., Nature; 2019, 572: 199-204). Cell clustering, based on similar transcriptomic profiles, identified the different cell types within the tissue (FIG. 3C). As indicated by their marked shift within the tSNE map, the cell population with the highest change in gene expression is the liver macrophage population (CD45⁺ MAF BZIP Transcription Factor B (MAFB⁺) cells) (FIG. 3C) Macrophages include a wide spectrum of different phenotypes from the classically activated pro-inflammatory macrophages (M1) to alternatively activated immunoregulatory macrophages (M2) (Krenkel and Tacke, Nature Rev., 2017, 17: 306-321). Characterization of macrophage marker gene expression revealed that the majority of Nizatidine-responding macrophages were characterized by a pro-inflammatory phenotype, as demonstrated by high levels of CLEC5A expression and low levels of CD163L1 and MARCO expression, two markers of immunoregulatory macrophages (FIG. 3D). Nizatidine treatment decreased macrophage CLEC5A expression (FIG. 3D), suggesting that Nizatidine modulates the phenotype of inflammatory macrophages. Furthermore, it was observed that Nizatidine decreases expression of the receptor sialic-acid-binding Ig-like lectin 10 (Siglec-10) in macrophages (FIG. 3D), a recently uncovered immune checkpoint shown to inhibit effector functions of immune cells in cancer (Barenwaldt and Laubli, Expert Opin. Ther. Targets, 2019, 23: 839-853).

Next, the present Inventors performed GSEA for differentially expressed genes after Nizatidine treatment in these macrophages. They observed that Nizatidine suppresses the pro-inflammatory macrophage (M1) signature but only partially induces the immunoregulatory macrophage (M2) signature (FIG. 3E) (Martinez et al., J. Immunol., 2006, 177: 7303-7311). Macrophages exhibit remarkable plasticity and can evolve in different subpopulations with atypical or intermediate profiles in response to environmental stimuli, sharing characteristic of more than one population (Krenkel and Tacke, Nature Rev., 2017, 17: 306-321). In accordance with this observation, the present Inventors showed that Nizatidine suppresses pro-inflammatory and pro-fibrogenic responses (i.e., TNFα, IL2 and IL6 signaling pathways) while augmenting IFNγ response and antigen processing and presentation, suggesting a shift from pro-inflammatory to an “atypical” immunoregulatory profile (FIG. 3F). Moreover, they observe a strong suppression of the c-Myc pathway expression, a major actor involved in the polarization of macrophages through a typical M2 profile (FIG. 3F) (Yang et al., Cell Death Dis., 2018, 9: 793).

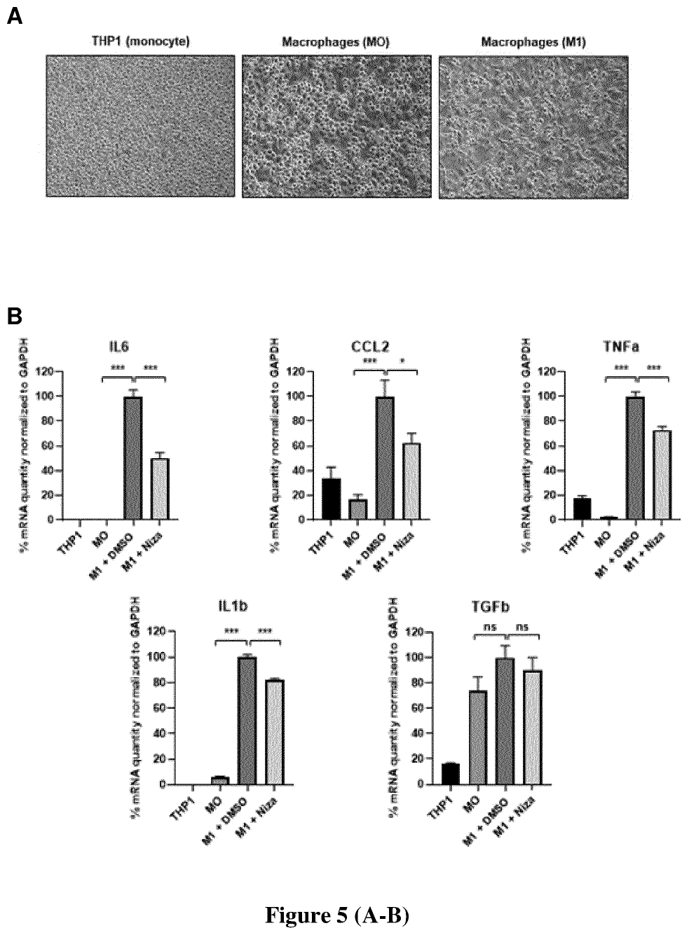

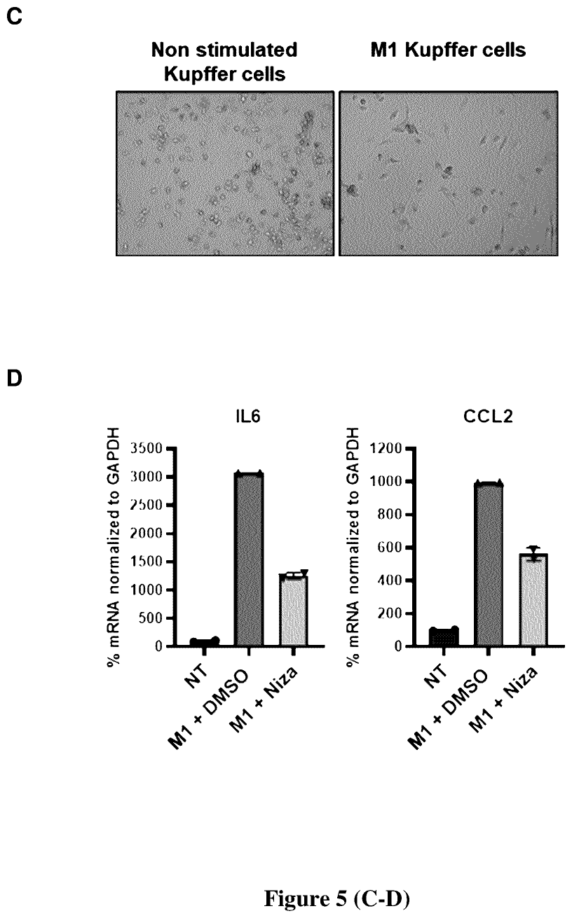

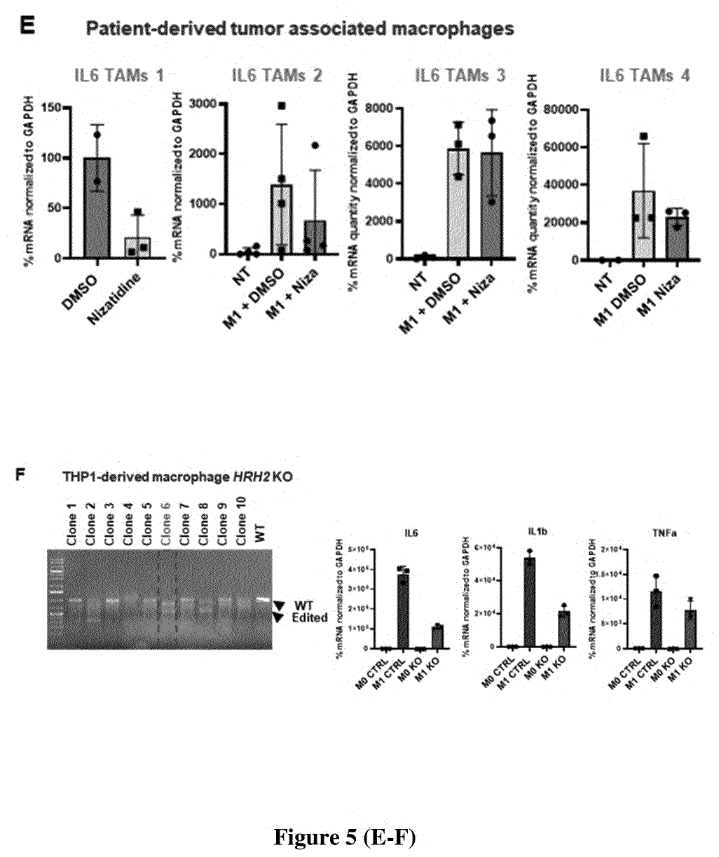

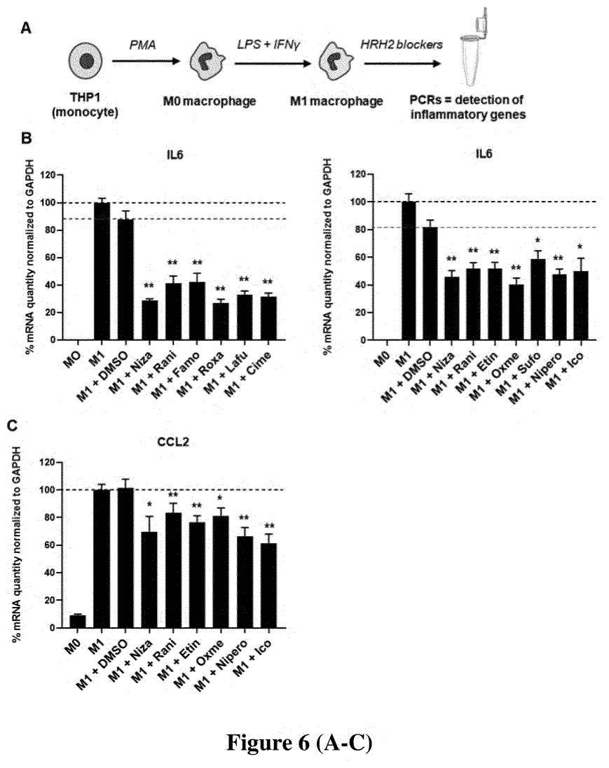

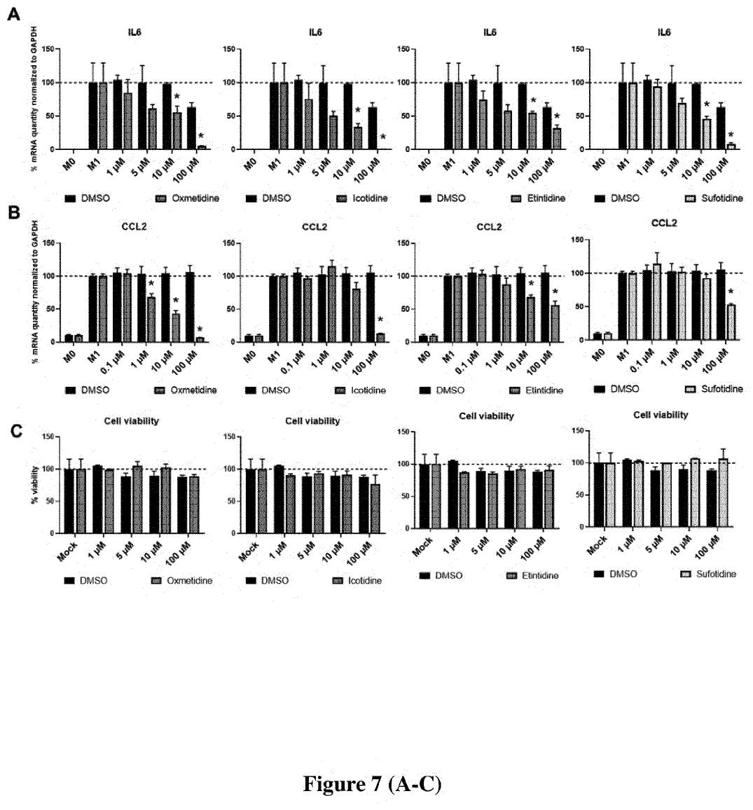

Gene-level analysis in macrophages also showed a decrease in neutrophil/monocyte chemoattractant and pro-inflammatory cytokine expression (i.e., CXCL5, CCL2, CCL5), as well as a suppression of pro-fibrotic/tumorigenic soluble factor expression (i.e., IL6, IL1b PDGF, MMP9, TNFα) (FIG. 3G) (Pello et al., Blood, 2012, 119: 411-421; Yang et al., Cell Death Dis., 2018, 9: 793). It is worth noting that a direct effect of Nizatidine treatment on pro-inflammatory cytokine expression was confirmed in THP1-derived M1-polarized macrophages (FIG. 5A-B), as well as in Kupffer cells isolated from patient tissues (FIG. 5C-D). The Inventors also observed a similar effect of other H2 blockers on pro-inflammatory cytokine expression (i.e., Oxmetidine, Icotidine, Etintidine, Sufotidine, Ranitidine, Famotidine) (FIGS. 6 and 7). Interestingly, the increase in major histocompatibility complex (MHC) molecules expression correlated with the restoration of antigen presentation-related pathways (FIG. 3G), suggesting an improvement of anti-cancer immunity. Collectively, these data suggest that nizatidine targets pro-inflammatory HRH2⁺, CLEC5A^{high}, MARCO^{low}, liver macrophages and modulates their phenotype, which in turn contributes to amelioration of liver disease and prevention of HCC.

Macrophage-Hepatocyte Crosstalk Activates HRH2 Signaling in Hepatocytes. To further investigate the functional role of both hepatocyte and macrophage HRH2 as a therapeutic target, the Inventors studied cellular crosstalk by incubating PHH with the supernatant of activated macrophages. As shown in FIG. 4C, the supernatant of activated macrophages resulted in modulation of hepatocyte signaling as shown by an increase in CREB5 expression (FIG. 4C). GalNac-RNAi-mediated HRH2 silencing resulting in an attenuation of CREB5 expression (FIG. 4C). These data suggest hepatocyte-macrophage crosstalk with activation of HRH2-dependent signaling in hepatocytes by histamine and cytokines released by pro-inflammatory macrophages. Macrophage-hepatocyte crosstalk may play an important role in the mechanism of action of HRH2-targeting agents for prevention and treatment of liver disease and cancer.

Nizatidine Improves Liver Inflammation and Immune Surveillance by Targeting Liver Macrophages. A direct effect of Nizatidine treatment on pro-inflammatory cytokine expression was confirmed in THP1-derived macrophages with similar effects of other HRH2 blockers as well as patient-derived Kupffer cells and patient-derived tumor macrophages (FIG. 5E). To address the functional role of HRH2 in macrophages by an additional experimental approach, the inventors have generated a HRH2 KO THP1-derived macrophage cell line and assessed its functional phenotype. Interestingly, the HRH2 KO resulted in a similar modulation of pro-inflammatory cytokines as treatment with Nizatidine (FIG. 5F). These data suggest that at least a part of the observed immunomodulatory effect of Nizatidine in THP1-derived macrophages is mediated through HRH2 and that macrophage HRH2 likely contributes to the therapeutic effect of Nizatidine in vivo.

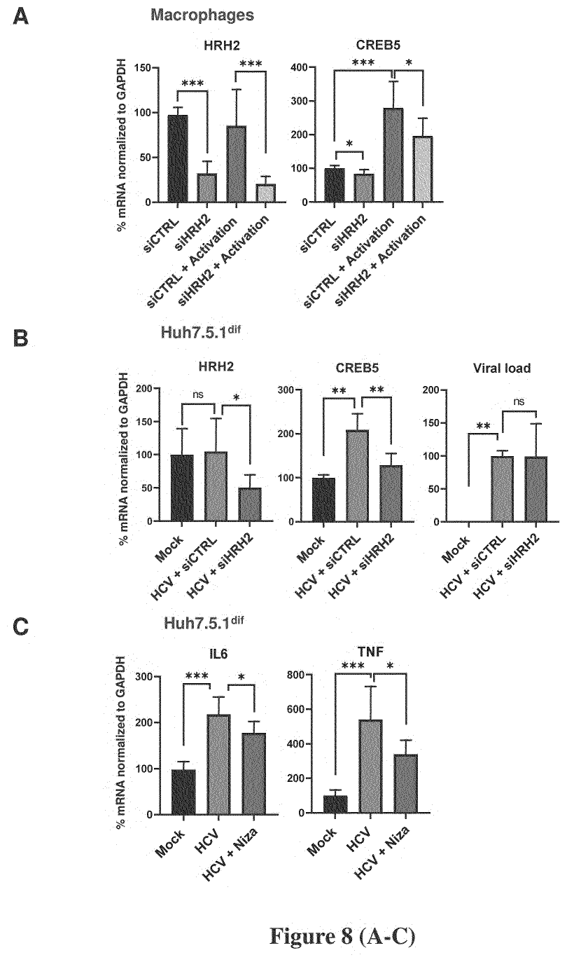

The cPLS System Models Nizatidine-Targeted Pathways in Both Parenchymal and Non-Parenchymal Liver Cells. Since Nizatidine was discovered using an HCV-infected transformed liver cancer cell line and mechanistic studies unraveled a dual mechanism of action on both hepatocytes and liver macrophages, the present Inventors investigated whether the cPLS model also recapitulates Nizatidine-target cell interactions in macrophages. The Inventors performed side-by-side loss-of-function studies of HRH2 in the cPLS system consisting of HCV-infected Huh7.5.1^{dif} cells and activated THP1-derived macrophages as a surrogate model for the perturbation of liver macrophages. In both cell types, CREB5 expression was similarly upregulated upon pathogenic insults and suppressed by HRH2 knock-down (FIG. 8A-B). Moreover, IL6 and TNFα expression were similarly increased upon pathogenic insults in macrophages and in the Huh7.5.1^{dif} cell-based system and decreased by Nizatidine treatment in both cell types (FIG. 8C and FIG. 5). Collectively, these data demonstrate that the cPLS system captures cellular signaling pathways targeted by Nizatidine in both hepatocytes and liver macrophages.

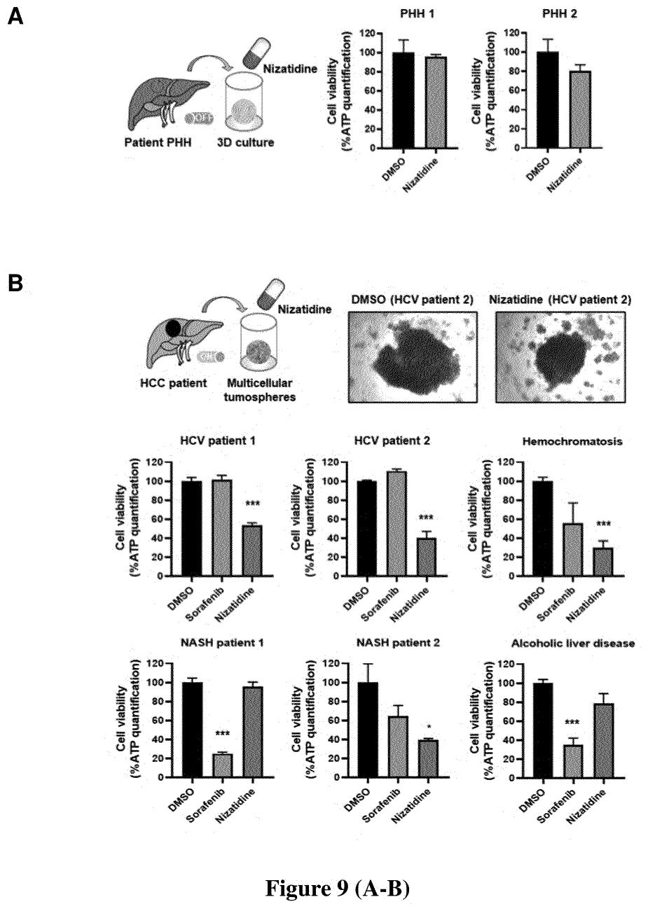

Nizatidine Decreases HCC Cell Viability in ex vivo Culture of Patient-Derived Precision-Cut Liver Tissues and Tumorspheroids. Finally, the present Inventors aimed to investigate the potential clinical efficacy of Nizatidine in advanced liver disease. To assess whether Nizatidine may also contribute to a direct effect on emerging HCC in advanced liver disease, they investigated the effect of Nizatidine in 3D patient-derived HCC tumor spheroids. In contrast to other 3D culture system, HCC spheroids include the tumor microenvironment, allowing establishment of cell-matrix and cell-cell contact between hepatocytes and non-parenchymal cells, including tumor-associated macrophages (Hendriks et al., Sci. Rep., 2016, 6:35434). While Nizatidine had no effect on PHH viability in line with its safe clinical profile (FIG. 9A), treatment with the compound resulted in a significant decrease of tumor spheroid viability in 4 out of 6 patients, independently of the etiology, including a pronounced effect on tumors that did not respond to sorafenib (FIG. 9B). Interestingly, Nizatidine-induced alteration of liver macrophage gene expression (FIG. 3) was associated with a robust ex vivo response to the drug in the same patient (FIG. 9B, HCV patient 2). Collectively, these data suggest that HRH2 targeting may have clinical efficacy in patients with advanced chronic liver disease and hepatobiliary cancer.

### DISCUSSION

The present Inventors discovered Nizatidine as a compound useful for the treatment of chronic liver disease and HCC prevention. While the PLS reversal/induction in a cell-based model is a simplification of the molecular processes observed in authentic chronic liver diseases, the validity of the drug discovery approach is supported by the identification of compounds with completed in vivo proof-of-concept for treatment of chronic liver disease and HCC prevention such as Nizatidine (this study), erlotinib (Fuchs et al., Hepatology, 2014, DOI 10.1002/hep.26898), pioglitazone (Li et al., Surg. Off. J. Soc. Surg. Aliment. Tract. 2019, 23: 101-111), the BRD4 inhibitor JQ1 (Juling et al., Gut 2020, 10.1136/gutjnl-2019-318918) or captopril—an ACE inhibitor with clinical efficacy for liver fibrosis and improvement of disease progression (Kim et al., Hepatol. Int., 2016, 10: 819-828).

The therapeutic effect of Nizatidine was found to be mediated by two complementary mechanisms: (1) Hepatocytes/parenchymal cancer cells: the functional data obtained in patient-derived primary cells demonstrate that the HRH2/CREB5 signaling pathway is perturbed in hepatocytes during liver injury. HRH2 inhibition by Nizatidine reverts HRH2 signaling as shown by modulation of HRH2 and CREB5 expression in hepatocytes in vivo. Furthermore, HRH2 KO significantly decreased the proliferation of a human HCC tumor cell line suggesting a direct functional role of the HRH2 pathway in tumor growth. The present findings are consistent with studies demonstrating that CREB5 overexpression in parenchymal cells is associated with tumor recurrence, metastasis, poor prognosis and overall survival (Abramovitch et al., Cancer Res., 2004, 64: 1338-1346; Chlabra et al., Oncol. Rep., 2007, 18: 953-958; He et al., Oncol., Lett., 2017, 14: 8156-8161; Westbom et al., Am. J. Pathol., 2014, 184: 2816-2827). CREB5 is a transcription factor belonging to the CREB protein family that regulates diverse cellular responses, including proliferation, survival, and differentiation. Upregulation of CREB protein can transform normal parenchymal cells into tumor cells through aberrant activation of downstream pathways such as growth factor receptor (i.e., EGFR) and cytokine/JAK/STAT pathways (Steven et al., Oncotarget, 2016, 7: 35454-35465). (2) Liver Macrophages: scRNA-Seq analyses in patient tissue uncovered HRH2+CLEC5A^{high} MARCO^{low} liver macrophages as the second target cell for nizatidine (FIG. 3). Clinical and experimental evidences have shown that macrophages play a key role in liver fibrosis progression (Ramachandran et al., Nature, 2019, 575; 512-518). Furthermore, some macrophages sub-populations enhance tumor progression by impairing cytotoxic LT CD8⁺ immune responses (Makarova-Rusher et al., J. Hepatol., 2015, 62: 1420-1429) and emerge as a target in cancer therapy (Barkal et al., Nature, 2019, 572: 392-396). Using scRNA-Seq analyses, they showed that Nizatidine enhanced INFγ-response pathways and pathways mediating antigen processing and presentation in macrophages. LT CD8⁺ cell responses in human HCC correlate with improved overall survival, longer relapse-free survival and diminished disease progression (for review see Ringelhan et al., Nature Immunol., 2018, 19: 222-232). Furthermore, they observed that Nizatidine treatment resulted in decreased expression of macrophage SIGLEC-10 (FIG. 3B). Activation of SIGLEC-10 by its ligands in macrophages induces a “don't eat me signal”, which blocks phagocytosis of transformed cells and has very recently been shown to contribute to immune invasion (Barkal et al., Nature, 2019, 572: 392-396). Therefore, it is conceivable that the decrease in SIGLEC-10 expression by Nizatidine may contribute to a restoration of anti-cancer immunity. The two target cells and mechanisms are most likely linked by hepatocyte-macrophage crosstalk.

Collectively, it was demonstrated that Nizatidine decreases the expression of pro-inflammatory, pro-fibrotic and pro-carcinogenic cytokines mediating liver disease progression and hepatocarcinogenesis. The data suggest that targeting HRH2+ macrophages may provide an opportunity to attenuate the fibrogenic and carcinogenic immune responses to liver injury while improving anti-cancer surveillance. The functional data obtained in macrophages (FIGS. 6-7) suggest a class-effect of HRH2 inhibitors. Interestingly, the histamine pathway has also been described to have a pathogenic role for primary sclerosing cholangitis and cholangiocarcinoma (Kennedy et al., Hepatol., 2018, doi: 10.1002/hep.29898). However, the mechanism in biliary disease is different from hepatocarcinogenesis by mast cells as a key driver for biliary disease biology. Of note, one animal model study has provided circumstantial evidence for an anti-HCC effect of Cimetidine (Furuta et al., Oncol. Rep., 2008, 19: 361-368). Furthermore, a clinical study in patients with transarterial chemoembolization has suggested that Cimetidine improves natural killer cell activity in peripheral blood (Nishiguchi et al., Hepatogastroenterology, 2003, 50: 460-462), which is different from the present findings obtained in liver tissue.

By improving liver inflammation, fibrosis and anti-cancer surveillance, HRH2 targeting compounds may provide a therapeutic approach for patients with chronic liver disease and/or HCC and/or CC and will guide future optimization of refined HRH2-targeting liver disease therapies. The excellent safety profile combined with robust therapeutic efficacy at doses which are achieved in patients treated with HRH2 antagonists, suggest rapid translatability of the approach.

### Example 2: Oxmetidine for the Treatment of Liver Fibrosis and Cancer Prevention

Materials and Methods. See above.

### Results

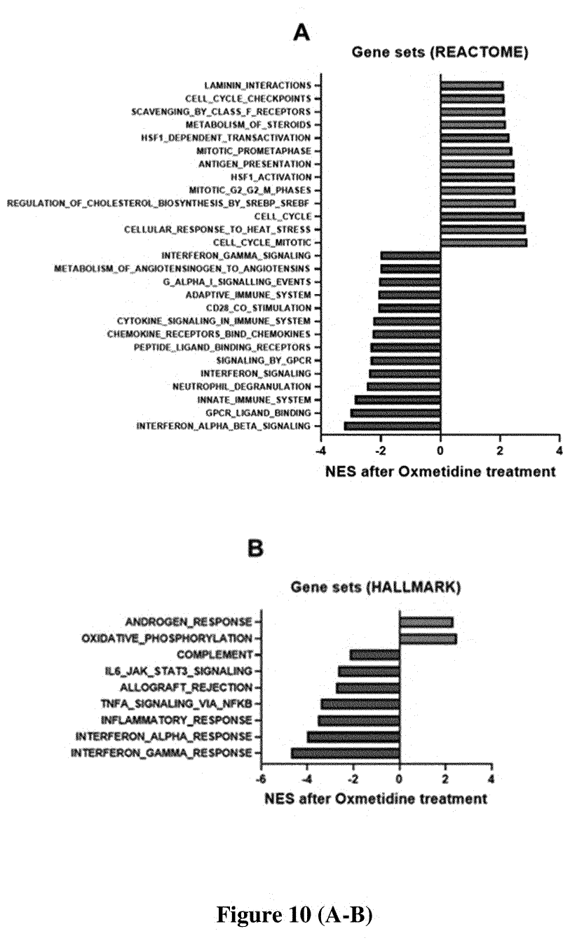

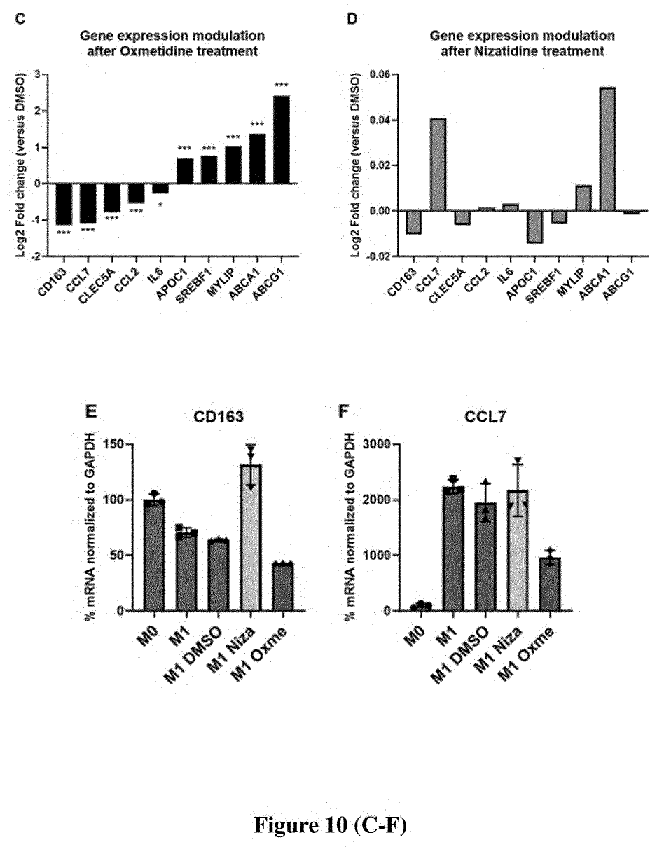

Oxmetidine Decreases Inflammation and Improves Metabolism in M1-polarized Macrophages. Previously, the present Inventors have identified liver macrophages as Nizatidine targets. They showed a direct effect of Nizatidine treatment on pro-inflammatory cytokine expression and on improvement of antigen processing and presentation process in macrophages. In order to assess the specific effect of Oxmetidine on macrophages, they performed RNA-Seq analysis on M1-polarized THP1-derived macrophages treated with Oxmetidine or DMSO as control (FIG. 10A-B). They observed that Oxmetidine strongly suppresses pathway mediating inflammation (i.e., TNFα, IL6) and improves antigen presentation related-pathways. In contrast to Nizatidine, they also observed a strong reduction in IFN response and an improvement of cell metabolism. This specific effect mediated by Oxmetidine was validated at the single gene level (top 10 of Oxmetidine modulated genes) showing a decrease in pro-inflammatory gene expression (i.e., CCL7, CCL2, IL6 . . . ) as well as an increase in expression of key genes regulating lipid metabolism in liver macrophages (i.e., ABCG1, ABCA1, SREBF1) (FIGS. 10C-F).

Together, these data demonstrate that Oxmetidine may improve liver inflammation, anti-cancer immunity and liver lipid metabolism by targeting HRH2⁺ macrophages. Moreover, the data presented herein suggest that Oxmetidine may have a superior therapeutic effect on liver disease and cancer compared to Nizatidine.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

1-19. (canceled)

20. A method of identifying an agent useful for the treatment or prevention of liver disease, the method comprising steps of:
providing a candidate compound; and
identifying the candidate compound as an agent useful for the treatment or prevention of liver disease if the candidate compound modulates the activity and/or function of a histamine 2 receptor.

21. The method according to claim 20, wherein the candidate compound is a histamine 2 receptor antagonist (H2 antagonist).

22. The method according to claim 21, wherein the candidate compound is a H2 antagonist in liver macrophages and/or in hepatocytes and/or in hepatocellular carcinoma cells.

23. The method according to claim 21, wherein the candidate compound is a selective H2 antagonist.

24. The method according to claim 21, wherein the candidate compound is selected from the group consisting of proteins, peptides, peptidomimetics, peptoids, polypeptides, saccharides, steroids, RNA agents, antibodies, ribozymes, antisense oligonucleotides, and small molecules.

25. The method according to claim 21, wherein the liver disease is selected from the group consisting of acute liver failure, liver fibrosis, alcohol-related liver disease, fatty liver disease (NASH, NAFLD), autoimmune liver disease, cirrhosis, genetic liver diseases, hepatitis and hepato-biliary cancers (HCC, CCA).

26. A method of identifying an agent useful for the treatment or prevention of liver disease, the method comprising steps of:
providing a candidate compound, wherein the candidate compound is a H2 antagonist; and
identifying the candidate compound as an agent useful for the treatment or prevention of liver disease if the candidate compound modulates the inflammatory profile of liver macrophages and/or of hepatocytes and/or hepatocellular carcinoma cell lines.

27. The method according to claim 26, wherein the candidate compound modulates the inflammatory profile of liver macrophages and/or of hepatocytes and/or hepatocellular carcinoma cell lines if the candidate compound decreases the overexpression of at least one pro-inflammatory cytokine or of at least one pro-fibrotic cytokine or soluble expression factor in liver macrophages and/or of hepatocytes and/or hepatocellular carcinoma cell lines.

28. The method according to claim 27, wherein the at least one pro-inflammatory cytokine is selected from the group consisting of IL6, IL1-a, IL1-b, IL-18, CCl2, CCL5, CXCL1, CXCL2, CXCL5, and TNF-a; and wherein the at least one pro-fibrotic cytokine or soluble expression factor is selected from the group consisting of TGF-b, PDGF, and MMP9.

29. The method according to claim 26, wherein the candidate compound is a selective H2 antagonist.

30. The method according to claim 26, wherein the candidate compound is selected from the group consisting of proteins, peptides, peptidomimetics, peptoids, polypeptides, saccharides, steroids, RNA agents, antibodies, ribozymes, antisense oligonucleotides, and small molecules.

31. The method according to claim 26, wherein the liver disease is selected from the group consisting of acute liver failure, liver fibrosis, alcohol-related liver disease, fatty liver disease (NASH, NAFLD), autoimmune liver disease, cirrhosis, genetic liver diseases, hepatitis and hepato-biliary cancers (HCC, CCA).

32. A method of identifying an agent useful for the treatment or prevention of liver disease, the method comprising steps of:
providing a candidate compound, wherein the candidate compound is a H2 antagonist; and
identifying the candidate compound as an agent useful for the treatment or prevention of liver disease if the candidate compound:
(a) decreases the expression of phosphorylated CREB1 and/or the expression of CREB5 in liver macrophages and/or hepatocytes and/or hepatocellular carcinoma cell lines; and/or
(b) decreases the expression of CLEC5A; and/or
(c) decreases the expression of SIGLEC-10 in liver macrophage; and/or
(d) decreases the expression of phosphorylated CREB1 and/or the expression of CREB5 in a human liver cancer cell line.

33. The method according to claim 32, wherein the candidate compound is a selective H2 antagonist.

34. The method according to claim 32, wherein the candidate compound is selected from the group consisting of proteins, peptides, peptidomimetics, peptoids, polypeptides, saccharides, steroids, RNA agents, antibodies, ribozymes, antisense oligonucleotides, and small molecules.

35. The method according to claim 32, wherein the liver disease is selected from the group consisting of acute liver failure, liver fibrosis, alcohol-related liver disease, fatty liver disease (NASH, NAFLD), autoimmune liver disease, cirrhosis, genetic liver diseases, hepatitis and hepato-biliary cancers (HCC, CCA).

36. A method for treating or preventing a liver disease in a subject, the method comprising a step of administering to the subject in need thereof an effective amount of a H2 antagonist, a chemical derivative thereof, a prodrug thereof, a pharmaceutically acceptable salt thereof, or a solvate thereof.

37. The method according to claim 36, wherein the H2 antagonist has been identified using the method according to claim 1.

38. The method according to claim 36, wherein the prodrug is a liver-targeted prodrug.

39. The method according to claim 36, wherein the H2 antagonist, chemical derivative thereof, prodrug thereof, pharmaceutically acceptable salt thereof, or solvate thereof is formulated with a liver-targeted drug carrier.

40. The method according to claim 39, wherein the H2 antagonist is a small molecule selected from the group consisting of Bisfentidine, Burimamide, Cimetidine, Dalcotidine, Donetidine, Ebrotidine, Etintidine, Famotidine, Icotidine, Impromidine Lafutidine, Lamtidine, Lavoltidine (Loxtidine), Lupitidine, Metiamide, Mifentidine, Niperotidine, Nizatidine, Osutidine, Oxmetidine, Pibutidine, Ramixotidine, Ranitidine, Ranitidine bismuth citrate, Roxatidine, Sufotidine, Tiotidine, Tuvatidine, Zaltidine, Zolantidine, AH-18801, AH-21201, AH-21272 SKF-93828, SKF-93996, AY-29315, BL-6341A (BMY-26539), BL-6548 (ORF-17910), BMY-25271, BMY-25368 (SKF-94482), BMY-25405, D-16637, DA-4634, FCE-23067, FRG-8701, FRG-8813, HB-408, HE-30-256, ICI-162846, ICIA-5165, IT-066 L-643441, L-64728, NO-794, ORF-17578 (BL-6217), RGW-2568, SR-58042, TAS, YM-14471, Wy-45086, Wy-45253, and Wy-45662, Wy-45727.

41. The method according to claim 40, wherein the H2 antagonist is Oxmetidine.

42. The method according to claim 36, wherein the liver disease is selected from the group consisting of acute liver failure, liver fibrosis, alcohol-related liver disease, fatty liver disease (NASH, NAFLD), autoimmune liver disease, cirrhosis, genetic liver diseases, hepatitis and hepato-biliary cancers (HCC, CCA).

43. The method according to claim 36, wherein the pharmaceutical composition comprises the H2 antagonist, chemical derivative thereof, prodrug thereof, pharmaceutically acceptable salt thereof, or solvate thereof, and at least one pharmaceutically acceptable excipient.
